(19) United States
(12) Patent Application Publication
(10) Pub. No.: US 2022/0313745 A1
(43) Pub. Date: Oct. 6, 2022

(54) WOUND TREATMENTS AND METHODS OF STABILIZING, PROTECTING, AND TREATING A WOUND

(71) Applicant: KERECIS HF, Isafjordur (IS)

(72) Inventor: Gudmundur Fertram SIGURJONSSON, Isafjordur (IS)

(21) Appl. No.: 17/704,539

(22) Filed: Mar. 25, 2022

Related U.S. Application Data
(60) Provisional application No. 63/166,064, filed on Mar. 25, 2021.
(60) Provisional application No. 63/166,005, filed on Mar. 25, 2021.

(51) Int. Cl. A61K 35/60; A61F 13/00

(52) U.S. Cl. CPC A61K 35/60; A61F 13/00017; A61F 13/00021; A61K 9/1682

(57) ABSTRACT

Wound treatments and wound treatment methods are provided that includes particles of decellularized fish skin. A predetermined percentage of at least a first portion of the particles of decellularized fish skin have a greatest dimension within a predetermined size threshold maximum and a minimum size threshold that is effective to preserve a matrix structure of the decellularized fish skin and to promote cellular regenerative ingrowth into a wound.

### CROSS-REFERENCE TO RELATED APPLICATIONS

This application claims the benefit of priority from U.S. provisional application No. 63/166,005, filed on Mar. 25, 2021, and U.S. provisional application No. 63/166,064, filed on Mar. 25, 2021, the entirety of these provisional applications being incorporated herein by reference.

### FIELD OF THE DISCLOSURE

The disclosure relates generally to wound treatments for stabilizing, protecting, and/or healing damaged tissue.

### BACKGROUND

Traumatic injuries, and wounds resulting therefrom, are an unsolved problem for healthcare providers and first responders, particularly in military and emergency situations, such as situations where there is a risk of significant blood loss, loss of limb, infection, and/or other trauma. Blast injuries from explosives are responsible for up to three-quarters of all field-related military injuries, as these often result in complicated soft-tissue losses that result in infection. Wounds resulting from, for example, explosive devices or gunshot wounds often cause such significant blood loss that conventionally tourniquets have been applied to control the blood flow (e.g., reduce or altogether stop blood flow), with a high attendant risk of tissue necrosis and amputation. If used alone, however, a tourniquet can lead to death of the downstream tissue and result in amputation. There is a clear need for an improvement over tourniquets in such situations.

Some wound treatments, such as on the battlefield or in first-responder emergency situations, include hemostatic agents such as clotting powder, hydrated aluminum silicate (e.g., kaolin), and chemical cauterizing agents (e.g., silver nitrate and trichloroacetic acid). Such hemostatic agents act in a variety of ways to slow or stop bleeding at a wound site. For example, some clotting powders incorporate granulated chitosan to slow and/or stop bleeding. Chitosan is derived from the chitin-rich shells of crustaceans, and its hemostatic activity is known to be the result of direct electrostatic interaction between the negatively charged cell membranes of erythrocytes and the positively charged chitosan—independent of classical coagulation pathways.

On the other hand, some hemostatic agents are dependent on classical coagulation pathways and act to hyperactivate blood clotting factors in the blood to decrease clotting time. Hydrated aluminum silicates, such as kaolin, are known to act in this manner. Chemical cauterizing agents cause wound closure in a destructive manner—through chemical reactions that fuse tissue.

Additional compounds can be administered in the field to help stabilize and/or protect traumatic wounds and treat and/or prevent potential complications stemming therefrom, including anti-inflammatory compounds, pain-relieving drugs, and antibiotic ointments. However, the currently available treatment options are centered on stabilizing the patient and fail to act to preserve tissue conditions.

Failure to preserve the tissue conditions of traumatic wounds often leads to tissue desiccation and deterioration. Further, many of the current treatments fail to provide an adequate barrier to subsequent infection and/or fail to confine the wound away from dirt and harmful pathogens. Further, current treatments are ill equipped for adaptation to prolonged field care. In short, there is a need for improved approaches to stabilizing and/or protecting wounds, particularly in the field, to preserve the patient and the wound for further, subsequent care.

In other contexts, including operating rooms, traumatic injuries, severe burns, and long-term wound care settings, such as with diabetic patients, existing approaches to wound care are often unsatisfactory at best. Negative-pressure wound therapy (“NPWT”), for example, is often performed after debridement and is used to promote blood flow to the wound, control edema, and reduce the presence of proteases, thus leading to increased granulation and revascularization of the wound bed.

NPWT is often disadvantageous, however, in that it is unable to accurately control applied pressure in geometrically challenging wounds or wounds near or at anatomically sensitive areas where adhesive seals are hard to obtain, bleeding (which may be difficult to assess due to obstruction from the dressing), skin irritation, infection, discomfort, ingrowth of granulation tissue into dressing materials, as well as technical issues. Further, NPWT machines typically are bulky and rely on electricity.

Another commonly prescribed treatment for wounds is hyperbaric oxygen therapy, consisting of exposing the patient to elevated pressure (2.0-2.5 atm) while breathing pure oxygen, which aims to oxygenate the wound bed so as to promote wound healing. This is thought to work by increasing the partial pressure of oxygen and forcing oxygen into the bloodstream to a greater degree than possible in normal conditions. While certain clinicians put much store by this approach, hyperbaric oxygen therapy is inherently expensive (requiring dedicated high-pressure equipment and trained staff), time-consuming (requiring 60-90 minute sessions daily for many days), and has known risks, including the risk of seizures.

Other traditional approaches to wound therapy include the “ladder approach,” which starts with stabilizing the wound bed and eventually including grafts and tissue transfer so as to effect functional and aesthetic outcomes. However, certain wounds, such as traumatic blast wounds, are often not suited to the traditional ladder approach, as tissue damage extends beyond the visible wound.

The traditional reconstructive ladder approach has been adapted into a “reconstructive elevator” by trauma surgeons, facilitating rapid advance through available techniques and prioritizing functional and aesthetic outcomes. This results, in some cases, in patients skipping simpler options in order to obtain the optimal outcome. More recently, however, treatment options have benefited from the introduction of new technologies such as dermal regenerative templates (“DRTs”) which enable more-efficient treatment of complex wounds using simpler techniques. However, certain existing DRT options are not ideal for prolonged field care (“PFC”) because of the potential for hematoma formation and clinical infection due to shear forces incurred during patient transfer.

Blast injuries and other wounds, such as large and complex wounds, are often problematic because of the lack of donor tissue for such trauma and due to the patient's stability. Further, in PFC and in first-responder situations in which time is of the essence, grafts may be impractical because they are difficult-to-obtain, time-consuming, and resource-intensive. There is accordingly a need for a new DRT technology that is not sensitive, as with existing approaches, to shear forces during PFC and/or first-responder situations.

Moreover, wound care is also complicated by the complexity of wound geometries. For examples, wounds may be incised wounds, lacerations, abrasions, punctures, avulsions, amputations, or combinations thereof, with tunneling wounds, undermining wounds, and cavities, and the specific nature of the injury, such as a blast, a traumatic accident, or otherwise, adding to the complexity.

Whereas existing approaches to wound therapy and treatment involve placing a sheet of material that has been cut to a particular size at the wound bed, there is a need for a wound treatment that can be easily configured to the particular geometry of a wound or wounds on a patient without cutting a sheet material to a specific size, which may be a combination of different types of wounds with different conditions and needs for different areas of the wound bed, while being configured to promote healing, for example regeneration and/or regrowth of tissue, by providing in embodiments a scaffold material or materials.

Wound care may be yet further complicated by the type of wound, for example diabetic foot ulcers (DFUs), venous leg ulcers (VFUs), surgical wounds, pressure ulcers (PUs), burns, traumatic wounds, combinations thereof, and others.

While certain existing approaches to wound care involve the use of, for example, human placental-based connective tissue matrix materials, these materials have innate limitations due to the limited availability of the placental feedstock and the safety of donating human-based tissue.

In view of the foregoing, the inventors have found there is need for a wound treatment that is rugged and robust, low weight, small, easy to transport and handle, has low dependency on external power or specialized equipment, is modular and interoperable with current approaches to care. Further, there is a need for a wound treatment that is resistant to shear forces. Additionally, the inventors have found there is a need for a wound treatment that is configured to more-easily and more-effectively accommodate the various geometries of different wound types. There is further a need for a wound treatment that is sustainable, scalable, and safe for human use.

### SUMMARY

A wound treatment is provided comprising particles of decellularized fish skin, the particles having a greatest dimension within a predetermined size threshold, and a minimum size threshold that is effective to preserve the matrix structure of the decellularized fish skin and to promote cellular regenerative ingrowth into a wound.

A wound treatment method is provided comprising: providing shredded, decellularized fish skin particles, by reducing a sheet of decellularized fish skin; applying the shredded, decellularized fish skin particles to a wound bed; and covering the wound bed with a dressing.

A wound treatment method is provided comprising particles of decellularized fish skin, the particles having a greatest dimension within a predetermined size threshold, and a minimum size threshold that is effective to preserve the matrix structure of the decellularized fish skin and to promote cellular regenerative ingrowth into a wound; applying the particles of decellularized fish skin a wound bed; and covering the wound bed with a dressing.

A method of providing a wound treatment is provided, comprising: providing one or more sheets of decellularized fish skin; and grinding the one or more sheets of decellularized fish skin into particles.

A wound treatment is provided comprising particles of decellularized fish skin, wherein a predetermined percentage of at least a first portion of the particles of decellularized fish skin have a greatest dimension within a predetermined size threshold maximum and a minimum size threshold that is effective to preserve a matrix structure of the decellularized fish skin and to promote cellular regenerative ingrowth into a wound.

A wound treatment method, the method comprising: providing particles of decellularized fish skin, a predetermined percentage of at least a first portion of the particles of decellularized fish skin having a greatest dimension within a predetermined size threshold maximum and a minimum size threshold that is effective to preserve a matrix structure of the decellularized fish skin and to promote cellular regenerative ingrowth into a wound; applying the particles of decellularized fish skin to a wound bed; and covering the wound bed with a dressing.

A method of providing a wound treatment, comprising: providing one or more sheets of decellularized fish skin; and shredding or grinding the one or more sheets of decellularized fish skin into particles of decellularized fish skin such that a predetermined percentage of at least a first portion of the particles of decellularized fish skin have a greatest dimension within a predetermined size threshold maximum and a minimum size threshold that is effective to preserve a matrix structure of the decellularized fish skin and to promote cellular regenerative ingrowth into a wound.

The wound treatment embodiments and treatment methods of the present disclosure advantageously solve one or more of the problems in the art of wound treatments for stabilizing, protecting, and/or healing a wound by providing a DRT that is more easily and/or effectively deployed in situations such as battlefields, first-responder situations such as car accidents, operating rooms, and other settings where wounds are treated. The wound treatment embodiments may advantageously be configured to resist shear forces, thereby facilitating transportation of a patient between one location, such as a battlefield, first-responder situation, or assisted-living environment to another location, such as a clinical setting, and vice versa.

Further, embodiments of the present disclosure additionally extend to bandages for treatment of a wound. An exemplary bandage can include or be configured to cooperate with shredded, decellularized fish skin particles. The shredded, decellularized fish skin particles can be rehydrated prior to application to a wound site. The bandage can also include a covering to secure the shredded, decellularized fish skin in particle form at a wound site, such as a deep wound where the shredded, decellularized fish skin particles can be compacted into the deep wound.

Accordingly, wound treatments, bandages, kits, and methods for stabilizing, protecting, and/or healing a wound are disclosed.

In embodiments, the shredded, decellularized fish skin particles may be used in combination with sheet-based decellularized fish skin scaffolds.

In other embodiments, a temporary wound treatment comprising comminuted decellularized fish skin in particle form is provided. Preferably, the particle form of comminuted decellularized fish skin is configured to minimize cellular scaffolding at a wound site during temporary wound treatment. This facilitates, in embodiments, a temporary wound treatment that stabilizes and/or protects a wound, for example preparatory to receiving subsequent or higher-level care. For example, the temporary wound treatment may be configured to protect and preserve a wound until a clinician removes the wound treatment for further treatment. By minimizing cellular scaffolding, the temporary wound treatment may be removed without exacerbating the wound or removing necessary cellular and vascular growth and structures.

In some embodiments, the temporary wound treatment additionally includes a temporary bandage configured and arranged to deliver the comminuted decellularized fish skin in particle form to a wound. The temporary bandage can include, for example, a contact layer configured to interface with a wound and to retain the comminuted decellularized fish skin at a wound and an outer cover associated with the contact layer and configured to retain the contact layer at a wound.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description that follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter.

### BRIEF DESCRIPTION OF THE DRAWINGS

FIG. 1A a plan view of a wound treatment according to an embodiment comprising shredded, decellularized fish skin particles in a first size.

FIG. 1B is a plan view of a wound treatment according to another embodiment comprising shredded, decellularized fish skin particles in a second size.

FIG. 1C is a plan view of a wound treatment according to another embodiment comprising shredded, decellularized fish skin particles in a third size.

FIG. 2A is a diagram of a method of treating a wound according to an embodiment of applying dry shredded, decellularized fish skin particles.

FIG. 2B is a diagram of a method of treating a wound according to an embodiment of applying moistened shredded, decellularized fish skin particles.

FIG. 3A is a perspective view of a prepared wound bed for being treated with a wound treatment according to an embodiment.

FIG. 3B is a perspective view of applying dry shredded, decellularized fish skin particles to the prepared wound bed of FIG. 3A.

FIG. 3C is a perspective view of the wound bed of FIG. 3A with the dry shredded, decellularized fish skin particles applied.

FIG. 4A is a perspective view of a package including dry shredded, decellularized fish skin particles with a liquid added thereto according to an embodiment of a wound treatment.

FIG. 4B is a perspective view of the package of FIG. 4A with the shredded, decellularized fish skin particles moistened with the liquid.

FIG. 4C is a perspective view of the package of FIG. 4A with the shredded, decellularized fish skin particles forming a paste.

FIG. 4D is a perspective view of a prepared wound for application of the paste of FIG. 4C.

FIG. 4E is a perspective view of the prepared wound to which the paste is applied with an applicator.

FIG. 4F is a perspective view of the prepared wound with the paste applied.

FIG. 5A is a perspective view of a prepared wound for application of a wound treatment according to an embodiment.

FIG. 5B is a perspective view of the prepared wound with the wound treatment applied.

FIG. 5C is a perspective view of the prepared wound with the wound treatment applied and a wound-treatment scaffold sheet applied.

FIG. 6A is a perspective view of a prepared wound for application of a wound treatment according to an embodiment.

FIG. 6B is a perspective view of the prepared wound with the wound treatment applied.

FIG. 6C is a perspective view of the prepared wound after application of the wound treatment.

FIG. 7A is a perspective view of a prepared wound for application of a wound treatment according to an embodiment.

FIG. 7B is a perspective view of the prepared wound with the wound treatment applied.

FIG. 7C is a perspective view of the prepared wound with the wound treatment applied and a wound-treatment scaffold sheet applied.

FIG. 8 illustrates a sample of decellularized fish skin scaffold material prior to comminution into particle form.

FIG. 9A illustrates various sized samples of decellularized fish skin scaffold material similar to that shown in FIG. 8.

FIG. 9B illustrates an exemplary depiction of large particles of comminuted decellularized fish skin resulting from grinding a sheet of decellularized fish skin scaffold material with a hemp grinder in accordance with embodiments of the present disclosure.

FIG. 9C illustrates an exemplary depiction of threaded, cotton-like fibers of comminuted decellularized fish skin resulting from grinding a sheet of decellularized fish skin scaffold material with a hemp grinder in accordance with embodiments of the present disclosure.

FIG. 9D is an exemplary depiction of small, powder-like particles of comminuted decellularized fish skin resulting from grinding a sheet of decellularized fish skin scaffold material with a hemp grinder in accordance with embodiments of the present disclosure.

FIG. 10 illustrates a schematic cross-section of a temporary bandage for use in retaining comminuted decellularized fish skin particles at a wound site to stabilize and/or protect said wound in accordance with exemplary treatments of the present disclosure.

FIG. 11 illustrates a schematic cross-section of a sleeve for use with comminuted decellularized fish skin in particle form to stabilize and/or protect a wound in accordance with exemplary treatments of the present disclosure.

FIG. 12 is an illustration of an exemplary kit, the components of which can be used to stabilize and/or protect a wound in accordance with embodiments of the present disclosure.

FIG. 13 illustrates a diagram of an exemplary method for stabilizing and/or protecting a wound using comminuted decellularized fish skin particles in accordance with embodiments of the present disclosure.

The drawing figures are not necessarily drawn to scale. Instead, they are drawn to provide a better understanding of the components and are not intended to be limiting in scope but to provide exemplary illustrations. The figures illustrate exemplary configurations of a wound treatment and features and sub-components thereof according to the present disclosure.

### DETAILED DESCRIPTION

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings described below. It should be understood, however, there is no intention to limit the disclosure to the specific embodiments disclosed, but on the contrary, the intention covers all modifications, alternative constructions, combinations, and equivalents falling within the spirit and scope of the disclosure.

The references used are provided merely for convenience and hence do not define the sphere of protection or the embodiments.

It will be understood that unless a term is expressly defined in this application to possess a described meaning, there is no intent to limit the meaning of such term, either expressly or indirectly, beyond its plain or ordinary meaning.

Any element in a claim that does not explicitly state “means for” performing a specified function, or “step for” performing a specific function is not to be interpreted as a “means” or “step” clause as specified in 35 U.S.C. § 112.

In embodiments, the wound treatment is or comprises particularized, particularly shredded, decellularized fish skin particles of at least one predetermined size. The particularized, i.e. shredded, decellularized fish skin particles are configured to provide a scaffold material for supporting cell migration, adherence, proliferation, and differentiation for facilitating the repair and/or replacement of tissue, as described in U.S. Pat. No. 8,613,957, granted on Dec. 24, 2013, the application of which was filed Oct. 6, 2010, the contents of which are incorporated by reference herein in its entirety.

The extracellular matrix (ECM) of vertebrates is a complex structural entity surrounding and supporting cells. ECM is composed of complex mixtures of structural proteins, the most abundant of which is collagen, and other specialized proteins and proteoglycans. The scaffold material described herein is a largely intact acellular scaffold of natural biological ECM components from fish skin. The scaffold can also comprise naturally occurring lipids from the fish skin. The native three-dimensional structure, composition, and function of the dermal ECM is essentially unaltered, and provides a scaffold to support cell migration, adherence, proliferation, and differentiation, thus facilitating the repair and/or replacement of tissue.

A scaffold material in accordance with this invention is obtained from intact fish skin. Any species of fish, including bony or cartilaginous fish, can be used as the source of the fish skin. For example, the source can be round fish like cod, haddock and catfish; flatfish, like halibut, plaice and sole; salmonids like salmon and trout; scombridaes like tuna; or small fish like herring, anchovies, mackerel and sardines. In certain embodiments the fish skin is obtained from cold-water oily fish and/or fish known to contain high amounts of omega-3 oil. Examples of fish high in omega-3 oil are salmon, pilchards, tuna, herring, cod, sardines, mackerel, sable fish, smelts, whitefish, hoki fish, and some varieties of trout.

The fish skin is removed from the fish before processing. If the fish skin is from a species of fish that has scales, the fish skin should be de-scaled so that a substantial portion of the scales are removed or at least the hydroxyapatite removed from the scales. The phrase “a substantial portion of the scales are removed” or “substantially scale-free” means that at least 95%, preferably at least 99%, and more preferably 100% of the scales on the fish skin are removed. “Substantially scale free” fish skin can also refer to fish skin from a fish species without scales. The scales are either removed prior to all processing, with purely mechanical pressure (via, e.g., knife, shaking with abrasives, water pressure, a special scale removal device that uses the same mechanical force as knives or other pressure device, like polishing with ceramic or plastic) or after some chemical treatment (e.g. decellularization) and then with mechanical pressure in order to wash the scales away. If the fish skin is first treated chemically and/or enzymatically (e.g. treatment with TRITON® X-100), the mechanical pressure generally needs to be gentle since the skin is more vulnerable to tearing after decellularization. The scales can be removed in more than one step, for example partial removal prior to decellularization followed by further removal during and/or after decellularization. Alternatively the scales can be removed by chemical treatment alone.

After the scales have been removed, the fish skin is optionally frozen prior to decellularization. The fish skin can be frozen quickly by incubating the skin in liquid nitrogen or using other special freezing equipment that can freeze the skin to −70° C. or lower, in order to preserve the collagen structure of the scaffold. Alternatively, the fish skin can be frozen in a conventional type of freezer that would be typically found in a fish factory. The freezing process may lyse or partially lyse the cells comprising the intact fish skin, and help facilitate decellularization of the fish skin. If the fish skin has been frozen, it can later be thawed for further processing.

Whether or not the fish skin was frozen, it can be washed with a buffer solution prior to further processing. For example, the fish skin can be washed 1-3 times with a buffer solution optionally containing one or more antioxidants (e.g. ascorbic acid (such as 50 mM ascorbic acid), Vitamins A, C, E, and beta carotene), antibiotics (e.g., streptomycin and penicillin), proteases (e.g. dispase II) and protease inhibitors (e.g. antipain, aprotinin, benzamidine, bestatin, DFP, EDTA, EGTA, leupeptin, pepstatin, phosphoramidon, and PMSF) to facilitate disinfection and stabilization of the fish skin. The buffer solution can be at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5. The buffer solution can also be used as a medium in which the fish skin can be stored for a few days up to a few weeks or more. In certain embodiments the fish skin is stored in the buffer solution at a temperature of about 4° C.

After freezing and/or washing and/or storage in a buffer solution, the fish skin is treated with one or more decellularizing solutions to remove cellular material, including antigenic material, from the fish skin with minimal to no damage to the mechanical and structural integrity and biological activity of the naturally occurring extracellular matrix.

The terms “extracellular matrix” or “ECM” as used herein refer to the non-cellular tissue material present within the fish skin that provides structural support to the skin cells in addition to performing various other important functions. The ECM described herein does not include matrix material that has been constituted or re-formed entirely from extracted, purified, or separated ECM components (e.g. collagen).

The terms “acellular,” “decellularized,” “decellularized fish skin,” and the like as used herein refer to a fish skin from which a substantial amount of cellular and nucleic acid content has been removed leaving a complex three-dimensional interstitial structure of ECM. In embodiments, “decellularized fish skin” may further entail fish skin which, in addition to the complex three-dimensional interstitial structure of ECM absent a substantial amount of cellular and nucleic acid content, includes omega 3 polyunsaturated fatty acids (PUFAs).

“Decellularizing agents” are those agents that are effective in removing a substantial amount of cellular and nucleic acid content from the ECM. The ECM is “decellularized” or “substantially free” of cellular and nucleic acid content (i.e. a “substantial amount” has been removed) when at least 50% of the viable and non-viable nucleic acids and other cellular material have been removed from the ECM. In certain embodiments, about 55%, 60%, 65%, 70%, 75%, 80%, 85%, 90%, 91%, 92%, 93%, 94%, 95%, 96%, 97%, 98%, 99%, 99.5%, or 100% of the viable and non-viable nucleic acids and cellular material are removed. Decellularization can be verified by, for example, testing the treated fish skin for DNA content. Removal of the nucleic acids from the ECM can be determined, for example, by histological examination of the ECM, and/or by a biochemical assay such as the PICOGREEN® assay, diphenylamine assay, or by PCR.

Decellularization disrupts the cell membranes and releases cellular content. Decellularizing may involve one or more physical treatments, one or more chemical treatments, one or more enzymatic treatments, or any combination thereof. Examples of physical treatments are sonication, mechanical agitation, mechanical massage, mechanical pressure, and freeze/thawing. Examples of chemical decellularizing agents are ionic salts (e.g. sodium azide), bases, acids, detergents (e.g. non-ionic and ionic detergents), oxidizing agents (e.g. hydrogen peroxide and peroxy acids), hypotonic solutions, hypertonic solutions, chelating agents (e.g. EDTA and EGTA), organic solvents (e.g. tri(n-butyl)-phosphate), ascorbic acid, methionine, cysteine, maleic acid, and polymers that bind to DNA (e.g. Poly-L-lysine, polyethylimine (PEI), and polyamindoamine (PAMAM)). Non-ionic detergents include 4-(1,1,3,3-Tetramethylbutyl)phenyl-polyethylene glycol, t-Octylphenoxypolyethoxyethanol, Polyethylene glycol tert-octylphenyl ether (TRITON® X-100) (Dow Chemical Co.). Ionic detergents include sodium dodecyl sulfate (SDS), sodium deoxycholate, TRITON® X-200, and zwitterionic detergents (e.g. CHAPS). Other suitable decullularizing detergents include polyoxyethylene (20) sorbitan mono-oleate and polyoxyethylene (80) sorbitan mono-oleate (Tween 20 and 80), 3-[(3-chloramidopropyl)-dimethylammino]-1-propane-sulfonate, octyl-glucoside and sodium dodecyl sulfate. Examples of enzymatic decellularizing agents are proteases, endonucleases, and exonucleases. Proteases include serine proteases (e.g. trypsin), threonine proteases, cysteine proteases, aspartate proteases, metalloproteases (e.g. thermolysin), and glutamic acid proteases. Decellularization is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

An example of a decellularization step is incubating the fish skin in a solution comprising 1 M NaCl, 2% deoxycholic acid, 0.02% sodium azide and 500 ppm streptomycin. In another example, the fish skin is incubated with a first decellularizing solution comprising a protease (e.g., 2.5 U/mL dispase II) and other components (e.g., 0.02% sodium azide). The first decellularizing solution is poured off and the fish skin is then treated with a second decellularizing solution such as a solution comprising a detergent (e.g., 0.5% TRITON® X-100) and other components (e.g. 0.02% sodium azide). In another example, the fish skin is first treated with a decellularizing solution comprising detergent (e.g. 0.5% TRITON® X-100) with other components (e.g. 0.02% EDTA, sodium azide, and/or deoxiholic acid), and then incubated in a second decellularizing solution comprising a detergent such as SDS.

The fish skin may or may not be incubated under shaking The decellularizing step(s) can be repeated as needed by pouring off any remaining decellularizing solution, optionally washing the fish skin with a buffer solution (e.g. Hank's Balanced Salt Solution), and then treating the fish skin again with another step of decellularization. Once a sufficient amount of cell material has been removed, the decellularizing solution can be removed (e.g., by aspiration or by gently pouring out the solution).

After decellularization, the fish skin can optionally be washed with water, buffer solution, and/or salt solution. Examples of suitable washing solutions include Dulbecco's phosphate buffered saline (DPBS), Hank's balanced salt solution (HBSS), Medium 199 (M199, SAFC Biosciences, Inc.) and/or L-glutamine. Washing step(s) are generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

The fish skin can optionally be bleached to improve the appearance of the final product. Bleaching can be carried out before, after, and/or concurrently with decellularization. For example, one or more bleaching agent can be incorporated into one or more of the decellularization solution(s) and/or into one or more buffer solution(s). Examples of bleaching agents include sodium sulfite, hydrogen peroxide, ammonium persulfate, potassium persulfate, and sodium persulfate. In certain embodiments, if a strong bleaching agent like persulfate(s) are used, bleaching and decellularization can be combined in a single step comprising incubating the fish skin in a mixture of one or more bleaching agents, thickeners, and peroxide sources. For example, a dry bleaching mixture can be prepared (see, e.g., the “bleaching mixtures” described in Example 5), followed by the addition of water, hydrogen peroxide, or a combination thereof to the dry mixture to form a bleaching solution that may also be sufficient for decellularization. The bleaching agents (e.g. sodium sulfite, hydrogen peroxide, ammonium persulfate, potassium persulfate, and sodium persulfate) should be about 40-60% w/w of the dry mixture. A combination of EDTA and persulfates may be added to the mixture to accelerate bleaching as well as decellularization.

In certain embodiments the concentration of EDTA in the dry mixture is about 0.25-5% w/w. Hydrogen peroxide can be about 15-25% of the mixture; the peroxide source can be sodium percarbonate and potassium percarbonate. Sodium phosphate perhydrate and sodium carbonate or magnesium metasilicate and silicium silicate can also be used as a peroxide source. The dry mixture can also include silica and hydrated silica, at for example 1-10% w/w, and optionally one or more stearate (e.g. ammonium stearate, sodium stearate, and/or magnesium stearate). In addition the dry mixture can optionally include thickeners, such as hydroxypropyl methylcellulose, hydroxyethylcellulose, algin (i.e. alginate), organic gums (e.g. cellulose, xanthan gum) sodium metasilicate, and combinations thereof to increase the viscosity of the bleaching/decellularization solution and protect protein fibers from damage. Bleaching, and/or bleaching plus decellularization, is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5. After bleaching and/or bleaching plus decellularization, the fish skin is optionally washed with a solution comprising L-glutamine under the pH conditions described above.

In certain embodiments, the fish skin is treated with a digestion enzyme. Similar to bleaching, digestion can be carried out before, after, and/or concurrently with decellularization. Suitable enzymes include proteases, for example serine proteases, threonine proteases, cysteine proteases, aspartate proteases, metalloproteases, and glutamic acid proteases. In certain embodiments the digestion enzyme is a serine protease such as trypsin. The digestion enzyme can be an enzyme that functions in an alkaline environment, limits cross-linking within the ECM, and softens the fish skin. Digestion is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

The decellularized fish skin can optionally be cryopreserved. Cryopreservation can involve immersing the fish skin in a cryoprotectant solution prior to freezing. The cryoprotectant solution generally comprises an appropriate buffer, one or more cryoprotectants, and optionally a solvent, e.g. an organic solvent which in combination with water undergoes minimal expansion and contraction. Examples of cryoprotectants include sucrose, raffinose, dextran, trehalose, dimethylacetamide, eimethylsulfoxide, ethylene glycol, glycerol, propylene glycol, 2-Methyl-2.4-pantandial, certain antifreeze proteins and peptides, and combinations thereof. Alternatively, if the decellularized fish skin is fast-frozen (flash-frozen) prior to sublimation in order to minimize ice crystals formed during the freezing step, the skins can optionally be frozen in a buffer solution that does not include cryoprotectants. Cryopreservation is generally carried out at a pH of at least 5.5, such as 6.0, 6.5, 7.0, 7.5, 8.0, 8.5, 9.0, 9.5, 10.0 or more. In certain embodiments the pH is between 7.0 and 9.0, e.g. between 7.5 and 8.5.

The decellularized fish skin can be packaged inside a sterile container, such as a glass vial or a pouch. In one embodiment, a TYVEK® pouch is used. For example, the fish skin can be incubated in a cryoprotectant solution, packaged in a TYVEK® pouch and then placed into a freeze dryer and frozen at a rate which is compatible with the cryoprotectant.

The decellularized fish skin can be lyophilized, i.e. frozen at a low temperature and under vacuum conditions so that water is removed sequentially from each ice crystal phase without ice re-crystallization. During lyophilization, water is generally removed first via sublimation and then via desorption if necessary. Another method of removing excess water after processing and before sterilization is vacuum pressing.

In certain embodiments, the decellularized fish skin is sterilized before and/or after being frozen. Sterilization methods are well known in the art. For example, the decellularized fish skin can be placed in an ethylene oxide chamber and treated with suitable cycles of ethylene oxide. Other sterilization methods include sterilizing with ozone, carbon dioxide, gaseous formaldehyde or radiation (e.g. gamma radiation, X-rays, electron beam processing, and subatomic particles).

As an alternative to or in addition to freezing, freeze-drying and/or vacuum pressing of water, the decellularized fish skin can be preserved in a non-aqueous solution such as alcohol.

The resulting product (scaffold material) is a sterile, collagen-based matrix that possesses properties that may facilitate the regeneration, repair and/or replacement of tissue (e.g., repair, regeneration, and/or growth of endogenous tissue). The term “scaffold material” refers to material comprising fish skin that has been decellularized and optionally bleached, digested, lyophilized, etc. as discussed above. The scaffold material can provide an intact scaffold for support of endothelial and/or epithelial cells, can be integrated by the host, is biocompatible, does not significantly calcify, and can be stored and transported at ambient temperatures. The phrase “integrated by the host” means herein that the cells and tissues of the patient being treated with the scaffold material can grow into the scaffold material and that the scaffold material is actually integrated/absorbed into the body of the patient. The term “biocompatible” refers to a material that is substantially non-toxic in the in vivo environment of its intended use, and that is not substantially rejected by the patient's physiological system (i.e., is non-antigenic).

This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part 1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

The scaffold material contains proteins from the extracellular matrix (ECM) of the fish skin. The ECM components in the scaffold material can include, for example, structural proteins; adhesive glycoproteins; proteoglycans; non-proteoglycan polysaccharides; and matricellular proteins. Examples of structural proteins include collagens (the most abundant protein in the ECM), such as fibrillar collagens (types I, II, III, V, and XI); facit collagens (types IX, XII, and XIV), short chain collagens (types VIII and X), basement membrane collagen (type IV), and other collagens (types VI, VII, and XIII); elastin; and laminin. Examples of adhesive glycoproteins include fibronectin; tenascins; and thrombospondin. Examples of proteoglycans include heparin sulfate; chondroitin sulfate; and keratan sulfate. An example of a non-proteoglycan polysaccharide is hyaluronic acid. Matricellular proteins are a structurally diverse group of extracellular proteins that regulate cell function via interactions with cell-surface receptors, cytokines, growth factors, proteases, and the ECM. Examples include thrombospondins (TSPs) 1 and 2; tenascins; and SPARC (secreted protein, acidic and rich in cysteine).

In certain embodiments, decellularization (and other optional processing steps) does not remove all of the naturally occurring lipids from the lipid layer of the fish skin. Thus, the scaffold material can comprise one or more lipids from the fish skin, particularly from the lipid layer of the fish skin. For example, the scaffold material may include up to about 25% w/w lipids (of dry weight of the total scaffold material after lyophilization), such as 0.1%, 0.5%, 1%, 2%, 3%, 4%, 5%, 6% 7%, 8%, 9%, 10%, 11%, 12%, 13%, 14%, 15%, 16%, 17%, 18%, 19%, 20%, 21%, 22%, 23%, or 24% w/w lipids. The presence of lipids in the scaffold material can be verified, for example, by organic solvent extraction followed by chromatography. Examples of suitable organic solvents include acetone and chloroform.

The lipids in the scaffold material can include, for example, fatty acyls (i.e. fatty acids, their conjugates, and derivates); glycerolipids; glycerophospholipids (i.e. phospholipids); sphingolipids; saccharolipids; polyketides; sterol lipids (i.e. sterols); certain fat-soluble vitamins; prenol lipids; and/or polyketides. Examples of fatty acyls include saturated fatty acids, such as polyunsaturated fatty acids; fatty esters; fatty amides; and eicosanoids. In certain embodiments the fatty acids include omega-3 fatty acids, such as eicosapentaenoic acid (EPA) and docosahexaenoic acid (DHA) (found in high concentration in fish oil). Other fatty acids found in fish oil include arachidic acid, gadoleic acid, arachidonic acid, butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, and lignoceric acid. Examples of glycerolipids include mono-, di-, and tri-substituted glycerols, such as monoacylglycerols, diacylglycerols, and triacylglycerols (i.e. monoglycerides, diglycerides, and triglycerides). Examples of glycerophospholipids include phosphatidylcholine; phosphatidylethanolamine; and phosphatidylserine. Examples of sphingolipids include phosphosphingolipids and glycosphingolipids. Examples of sterol lipids include cholesterol; steroids; and secosteroids (various forms of Vitamin D). Examples of prenol lipids include isoprenoids; carotenoids; and quinones and hydroquinones, such as Vitamins E and K.

The scaffold material can contain one or more added active agents (i.e. an agent that is added during or after processing of the scaffold material), such as antibiotics, antiseptics, antimicrobial agents, antivirals, antifungals, antiparasitics and anti-inflammatory agents. The active ingredient can be a compound or composition that facilitates wound care and/or tissue healing such as an antioxidant, or drug. It can also be a protein or proteins and/or other biologics. Antibiotics, antiseptics, and antimicrobial agents can be added in an amount sufficient to provide effective antimicrobial properties to the scaffold material. In certain embodiments, the antimicrobial agent is one or more antimicrobial metal, such as silver, gold, platinum, copper, zinc, or combinations thereof. For example, silver may be added to the scaffold material during processing in ionic, metal, elemental, and/or colloidal form. The silver may also be in combination with other antimicrobials. Anti-inflammatory agents can be added in an amount sufficient to reduce and/or inhibit inflammation at the wound or tissue area where the scaffold material is applied.

The scaffold material can be used in dried form. Alternatively, the scaffold material can be rehydrated prior to use. In certain embodiments, one or more scaffold materials are laminated together to form a thicker scaffold material.

Generally, the scaffold material is from about 0.1 to 4.0 mm thick (i.e. in cross-section), such as 0.5, 1.0, 1.5, 2.0, 2.5, 3.0 or 3.5 mm thick. The thickness can depend on a number of factors, including the species of fish used as the starting material, processing, lyophilization, and/or rehydration. Of course, the thickness is proportionately greater when the product comprises more than one layer of scaffold material.

The shredded, decellularized fish skin particles of wound treatment and method embodiments advantageously provide a sterile, collagen-based matrix that possesses properties that may facilitate the regeneration, repair, and/or growth of tissue, such as endogenous tissue, while being configured to be formed or added to a wound so as to better accommodate the geometry of a wound. In embodiments, the shredded, decellularized fish skin particles are configured to be packed into a wound, such as an undermined or tunneling wound, in ways that are not available using sheet-based materials. That is, the shredded, decellularized fish skin particles may be configured to promote integration, that is in which the cells and tissues of the patient being treated with the scaffold material can grow into the scaffold material and that the scaffold material is actually integrated/absorbed into the body of the patient.

Shredded, decellularized fish skin particles according to embodiments may, in embodiments, be configured for actively promoting wound healing as a physical scaffold for infiltrating cells involved in wound healing/repair, such as for cell ingrowth and neovascularization. The shredded, decellularized fish skin particles of wound treatment embodiments are configured to advantageously retain the three-dimensional (“3D”) structure of the decellularized fish skin with an Extracellular Matrix (“ECM”) that is recognizable, for instance, on a histology analysis. The dimensions of the shredded decellularized fish skin particles may additionally be configured so as to facilitate molding, packing, or otherwise applying the shredded decellularized fish skin particles into a wound cavity with greater precision than existing approaches to wound therapy.

In embodiments, the shredded, decellularized fish skin particles have a greatest dimension within a predetermined maximum size threshold and a minimum size threshold that is effective to preserve the matrix structure of the decellularized fish skin and to promote cellular regenerative ingrowth into a wound. That is, a greatest dimension, such as a greatest one of a length, width, and/or thickness of the shredded, decellularized fish skin particles, may be lower than a maximum size, such as 1 mm, and larger than a minimum size, such as a size at which the ECM is destroyed. In embodiments, the shredded, decellularized fish skin particles are obtained by providing a sheet of decellularized fish skin as described above and then shredding the sheet of decellularized fish skin and optionally sieving the shredded particles until the shredded, decellularized fish skin particles are within the predetermined minimum and maximum size thresholds.

The shredded, decellularized fish skin particles may further be configured to resist shear forces on account of their dimensions, thus allowing the shredded, decellularized fish skin particles to provide an improved wound treatment for patients who move or are moved between locations or settings, or during the normal course of activities by a patient, such as walking, during recovery.

The shredded, decellularized fish skin particles of embodiments may advantageously be topically applied to and/or implanted in a wound to provide a scaffold for cell ingrowth and neovascularization, with additional benefits including tissue scaffolding benefits, such as adhesion barrier, soft tissue repair, dehiscence prevention, and others.

Embodiments of the present disclosure additionally include kits for stabilizing, protecting, and/or healing a wound. An exemplary kit can include a container including shredded, decellularized fish skin in particle form for placement on or in a wound and to be retained at a wound site by a contact element. The shredded, decellularized fish skin particles may be configured for being placed on the wound bed in a dry form or in a wet form.

In some embodiments, the container further includes a contact element configured to interface with a wound and to retain the shredded decellularized fish skin in particle form at a wound and an outer cover configured to retain the contact element and the shredded decellularized fish skin in particle form at a wound. The container can additionally include a base material for carrying the shredded decellularized fish skin in particle form and for associating with the contact element. The container may also include one or more therapeutics comprising analgesics, anesthetics, cytokines, growth factors, hemostatic agents, antibiotics, antifungals, hydrating compounds, or combinations thereof.

The container may further comprise a liquid, such as a saline solution of a predetermined concentration, allowing a clinician to moisten the shredded, decellularized fish skin particles prior to or during application of the wound so as to apply the shredded, decellularized fish skin particles in wet form alternatively or in addition to application in dry form.

Embodiments of the present disclosure additionally extend to methods for stabilizing, protecting, and/or healing a wound. An exemplary method can include applying shredded decellularized fish skin in particle form to a wound.

Methods of applying shredded decellularized fish skin particles may include steps of preparing a wound bed, such as by removing necrotic tissue to obtain a clean wound surface and/or irrigating to remove debris and exudate; if applying the shredded decellularized fish skin particles in dry form, pouring or sprinkling the shredded decellularized fish skin particles onto the wound bed; if applying the shredded decellularized fish skin particles in wet form, moistening the shredded decellularized fish skin particles with a liquid, such as saline, and then applying the moistened shredded decellularized fish skin particles to the wound bed using a finger, tongue depressor, or other surgical implement; and covering and securing the wound bed with a dressing, such as a non-adherent dressing, foam, gauze, wrap, and/or other components as necessary.

In embodiments, applying shredded, decellularized fish skin particles to a wound provides scaffold material or materials to promote ingrowth of cellular tissue within the shredded, decellularized fish skin particles. In embodiments, the shredded, decellularized fish skin particles, when applied to a wound, resist shear forces.

In embodiments, a clinician may elect to apply the shredded, decellularized fish skin particles of wound treatment embodiments in dry form so as to allow the clinician to pour the shredded, decellularized fish skin particles onto the wound bed and to obtain a desired dispersion of particles. In other embodiments, a clinician may elect to apply the shredded, decellularized fish skin particles in wet form so as to allow the clinician to mold the resulting paste to the specific dimensions of the wound bed. In embodiments, a combination of wet and dry application may be used. In other embodiments, a combination of sizes of the shredded, decellularized fish skin particles may be used.

As used herein, the term “treatment” is intended to be understood by its common dictionary definition. That is, the term “treatment” broadly includes medical care and/or medicaments given to a patient for an illness or injury. As should be appreciated by those having skill in the art, a “treatment” includes the use of a chemical, physical, or biological agent to preserve or give particular properties to something. Thus, a “treatment” may be the medical care provided (i.e., in the form of a method or series of prescribed acts), or it may refer to the medicament used to preserve or give a particular property to something.

As a non-limiting example, the particle form of decellularized fish skin disclosed herein can be referred to as a “treatment”—i.e., a medicament used to preserve and/or stabilize a wound or which can provide any of the other disclosed beneficial effects to a wound site. Similarly, in some instances, a treatment includes use of the disclosed decellularized fish skin in particle form within methods for stabilizing and/or protecting a wound.

The terms “decellularized,” “decellularized fish skin,” “acellular fish skin,” and the like as used herein refer to a fish skin made according to any method and includes any embodiment disclosed in U.S. Pat. No. 8,613,957, titled, “Scaffold Material for Wound Care and/or Other Tissue Healing Applications,” which is incorporated herein by reference in its entirety. Accordingly, the terms “decellularized,” “decellularized fish skin,” “acellular fish skin,” and the like as used herein include descaled fish skin from which a substantial amount of cellular and nucleic acid content has been removed, leaving a complex three-dimensional interstitial structure of native extracellular matrix material (ECM). In general, the decellularization described above is a gentler form of processing than is otherwise required and/or routinely performed on mammalian tissues, which often utilize harsh chemical treatments and/or storage in chemicals (e.g., antibiotics).

The decellularization methods described in U.S. Pat. No. 8,613,957 result in production of a scaffold material that maintains a three-dimensional structure of natural extracellular matrix components, and this allows, in some instances, a physical medium by which stem cells—and other cells contributing to the wound healing process—may migrate across and/or be supported on to promote wound healing. The native structure of extracellular components, such as collagen, is maintained within the decellularized fish skin scaffolding material in addition to other native components such as Omega 3 polyunsaturated fatty acids (PUFAs).

Other scaffold materials that are derived from mammalian skin/membranes, such as placental-based wound treatments, are subjected to harsh chemical treatments due to concerns with viral and prion transmission risk and risks of allergic or other immune responses from the use of mammalian scaffold materials in humans. These treatments obliterate the natural three-dimensional configuration of extracellular components or otherwise render them inoperable to promote wound healing to the same or similar extent as decellularized fish skin.

On the other hand, disease transmission risk from the Atlantic cod (Gadus morhua)—and many other fish species—to humans is non-existent or at least far less probable. Additionally, decellularized fish skin likely does not contain allergenic components, significantly decreasing the risk of allergic or other immune response. Owing to the decreased risk of disease transmission and allergic response, decellularized fish skin is subjected to gentle processing that retains the biological structure and bioactive compounds of the extracellular matrix. Accordingly, decellularized fish skin is denuded of skin cells during processing, but it maintains the natural three-dimensional structure of extracellular components, which provides a natural scaffold to promote wound healing. In contrast, mammalian scaffold materials lack a three-dimensional structure and have lost other natural extracellular components and fail to promote wound healing in the same manner or to the same degree as decellularized fish skin.

Reconstituted collagen materials that are harvested through harsh physical and chemical treatments also fail to maintain their native three-dimensional structure, particularly within the natural context of other natural extracellular components. Similar to the mammalian-derived scaffold material discussed above, the lack of a native structure and/or three-dimensional extracellular matrix environment provided by reconstituted collagen materials renders them unable to provide a comparable or analogous scaffold for promoting wound healing.

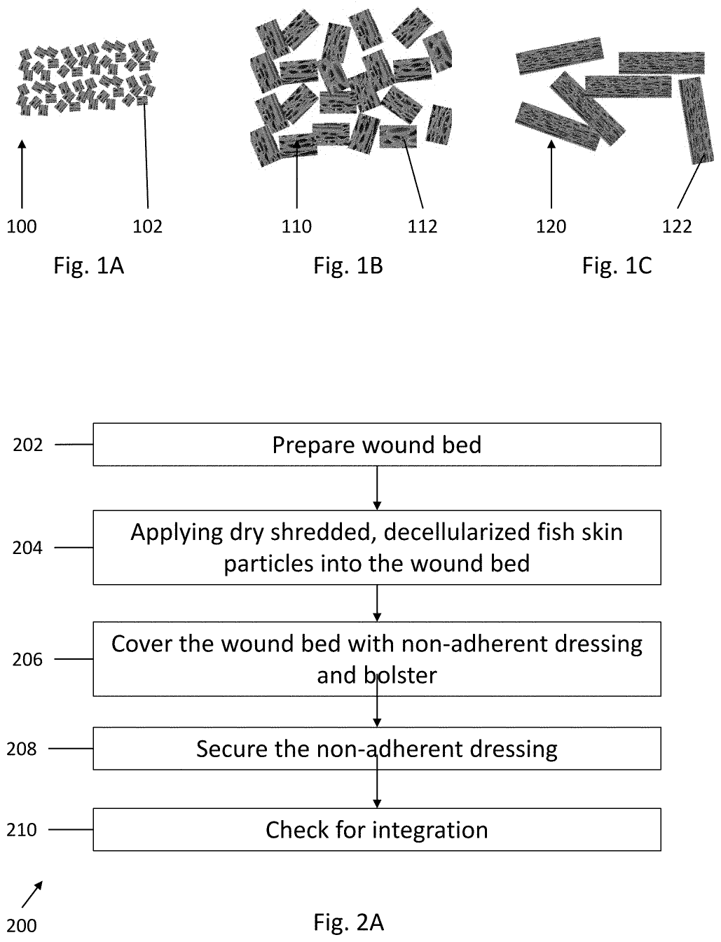

Referring to the embodiment of FIGS. 1A-1C, a wound treatment 100, 110, 120 according to embodiments of the present disclosure are shown and described. The wound treatments 100, 110, 120 comprise one or more shredded, decellularized fish skin particles 102, 112, 122.

In embodiments, the shredded, decellularized fish skin particles have a greatest dimension within a predetermined maximum size threshold and a minimum size threshold that is effective to preserve the matrix structure of the decellularized fish skin and to promote cellular regenerative ingrowth into a wound. That is, a greatest dimension, such as a greatest one of a length, width, and/or thickness of the shredded, decellularized fish skin particles, may be lower than a maximum size, such as 1 mm, and larger than a minimum size, such as a size at which the ECM is destroyed.

In embodiments, the shredded, decellularized fish skin particles are obtained by providing a sheet of decellularized fish skin as described above and then shredding or grinding the sheet of decellularized fish skin and in embodiments sieving the shredded particles until the shredded, decellularized fish skin particles are entirely or substantially within the predetermined minimum and maximum size thresholds. In embodiments, the shredded, decellularized fish skin particles are substantially within the predetermined minimum and maximum size thresholds when approximately 75% or more of the particles have a greatest dimension that falls between the predetermined minimum and maximum size thresholds, in embodiments when approximately 90% or more of the particles have a greatest dimension that falls between the predetermined minimum and maximum size thresholds, in embodiments when approximately 95% or more of the particles have a greatest dimension that falls between the predetermined minimum and maximum size thresholds, or other percentages as suitable.

The shredded, decellularized fish skin particles 102 are advantageously less than 1 mm in size. That is, one or more of a thickness, length, width, or other measurement of the particularized decellularized fish skin particles 102, i.e. a greatest one of the thickness, length, and width is less than 1 mm. In embodiments, the particularized decellularized fish skin particles 122 are, on average or according to any suitable predetermined threshold, less than 1 mm. In other embodiments, all of the particularized decellularized fish skin particles 122 are less than 1 mm.

In embodiments, the particularized decellularized fish skin particles 102 are no more than 1 mm in any respect. This advantageously allows maximum moldability of the particularized decellularized fish skin particles so as to fit snugly within a wound.

In other embodiments, particularized decellularized fish skin particles 112 are between 1 and 2 mm in size. That is, one or more of a thickness, length, width, or other measurement of the particularized decellularized fish skin particles 102, i.e. a greatest one of the thickness, length, and width is less than 2 mm and greater than 1 mm. The particularized decellularized fish skin particles 112 are, due to their size, advantageously able to retain ECM structure and still be handled easily by a practitioner, such as a clinician.

In another embodiment, the particularized decellularized fish skin particles 122 are greater than 2 mm in size. That is, one or more of a thickness, length, width, or other measurement of the particularized decellularized fish skin particles 122 is greater than 2 mm, i.e. a greatest one of the thickness, length, and width is greater than 2 mm. In embodiments, the particularized decellularized fish skin particles 122 are, on average or according to any suitable predetermined threshold, greater than 2 mm. In other embodiments, all of the particularized decellularized fish skin particles 122 are greater than 2 mm. In another embodiment, the predetermined size threshold does not pertain to a thickness of the particles of the decellularized fish skin.

The particularized decellularized fish skin particles 102, 112, 122 may be obtained, in embodiments, by grinding a sheet-based decellularized fish skin material to the a desired size using a suitable grinding device, for example a Universal Cutting Mill Pulverisette 19 with a variable speed of 50-700 rpm, available from Fritsch GmbH of Idar-Oberstein, Germany.

The grinding device or the processing of the fish skins may comprise use of one or more sieves or filters configured to separate the ground sheet-based decellularized fish skin material into suitable size distributions. For example, the sieves may capture shredded particles greater than a predetermined maximum size threshold while allowing smaller shredded particles to pass through to a receptacle. A second sieve may be used to capture shredded particles greater than a predetermined minimum size threshold while allowing smaller shredded particles to pass through to a second receptacle, such as a waste receptacle.

In certain embodiments, the shredded, decellularized fish skin particles may have a size distribution of between 2 mm and 2.8 mm (i.e. a greatest of one or more of a thickness, length, width, or other measurement of the shredded, decellularized fish skin particles 122 is between 2 mm and 2.8 mm), between 1.4 and 1.99 mm (i.e. a greatest of one or more of a thickness, length, width, or other measurement of the shredded, decellularized fish skin particles 122 is between 1.4 mm and 1.99 mm), between 1 mm and 1.39 mm (i.e. a greatest of one or more of a thickness, length, width, or other measurement of the shredded, decellularized fish skin particles 122 is between 1 mm and 1.39 mm), greater than 1 mm (i.e. a greatest of one or more of a thickness, length, width, or other measurement of the shredded, decellularized fish skin particles 122 is greater than 1 mm), or any other suitable dimensions.

In embodiments of a wound treatment and associated methods, a combination of two or more different sizes of shredded, decellularized fish skin particles is provided in suitable proportions of the sizes or thresholds described herein, such as 50:50 of a first size and a second size by volume or weight, 60:40 of a first size and a second size by volume or weight, 70:30 of a first size and a second size by volume or weight, 25:75 of a first size and a second size by volume or weight, 80:20 of a first size and a second size by volume or weight 90:10 of a first size and a second size by volume or weight, or 95:5 of a first size and a second size by volume or weight, 97:3 of a first size and a second size by volume or weight, 98:2 of a first size and a second size by volume or weight, or 99:1 of a first size and a second size by volume or weight, or any other suitable proportions.

Further, in embodiments of a wound treatment and associated methods, a combination of three or more different sizes of shredded, decellularized fish skin particles is provided in suitable proportions of the sizes or thresholds described herein, such as 50:25:25 of a first size, a second size, and a third size by volume or weight, 60:20:20 of a first size, a second size, and a third size by volume or weight, 70:15:15 of a first size, a second size, and a third size by volume or weight, 80:10:10 of a first size, a second size, and a third size by volume or weight, 90:5:5 of a first size, a second size, and a third size by volume or weight, 60:30:10 of a first size, a second size, and a third size by volume or weight, 70:20:10 of a first size, a second size, and a third size by volume or weight, 90:9:1 of a first size, a second size, and a third size by volume or weight, or any other combinations.

In one or more embodiments, the anti-viral and anti-bacterial properties of shredded, decellularized fish skin particles act to prevent bacterial and/or viral infection at the wound site, thereby decreasing potential complications (e.g., infection of the wound) and/or increasing the diversity of applications and or circumstances where wound treatment and method embodiments according to the present disclosure may be used.

Additionally, the anti-inflammatory (or inflammatory regulating) properties of Omega 3 PUFAs within shredded, decellularized fish skin particles help regulate inflammation at the wound site, which in some embodiments helps to stabilize, protect, and/or heal the tissue.

That is, the Omega 3 PUFAs are found within fish skin and are retained after the decellularization treatment. Previous studies have shown that Omega 3 PUFAs possess anti-viral and anti-bacterial properties and also act as regulators of inflammation. Shredded, decellularized fish skin particles inherit and retain these salubrious properties after processing as described herein, further contributing to the healing properties of the shredded, decellularized fish skin particles of the wound treatment and associated method embodiments.

In some embodiments, the kits (or components thereof) can be used for stabilizing, covering, and/or initializing the wound healing process in tunneled/undermined wounds or other traumatic wounds, including, for example, for the local management of bleeding wounds (e.g., cuts, lacerations, and abrasions) and/or the temporary management of severely bleeding or hemorrhaging wounds.

In some embodiments, additional advantages can be realized through the utilization of systems, kits, and/or methods that incorporate the two types of decellularized fish skin products (e.g., sheet-based and shredded, decellularized fish skin) together for complicated soft-tissue wounds. These two types of fish skin can be used in combination with each, serving a different application purpose.

For example, deep, asymmetric, and undermined wounds can be filled with shredded, decellularized fish skin before being secured with sheets for optimal wound healing, bleeding control, and protection against infection during transit to a higher-Echelon facility. The secondary cover with sheet-based decellularized fish skin sheets protects the shredded, decellularized fish skin particle-based wound treatment during dressing changes, such as from shear forces, and adds a bacterial and hemostatic barrier during transit. Consequently, injured individuals can begin healing while they await extraction to a healthcare facility, resulting in better-quality wound beds for subsequent grafting.

Additionally, kits having shredded, decellularized fish skin particles can provide one or more surgical benefits, including, for example: providing wounds an initial treatment approach that will control bleeding, stabilize the wound bed, begin the skin regeneration process, and provide microbial control; simplify the treatment options for tunneling and undermining wounds that traditional materials are not physically optimized to address; fill deep sacral and pressure wounds, allowing smaller flaps to be applied and improving the likelihood of flap success; and temporize wounds in preparation for autografting and/or skin flap creation.

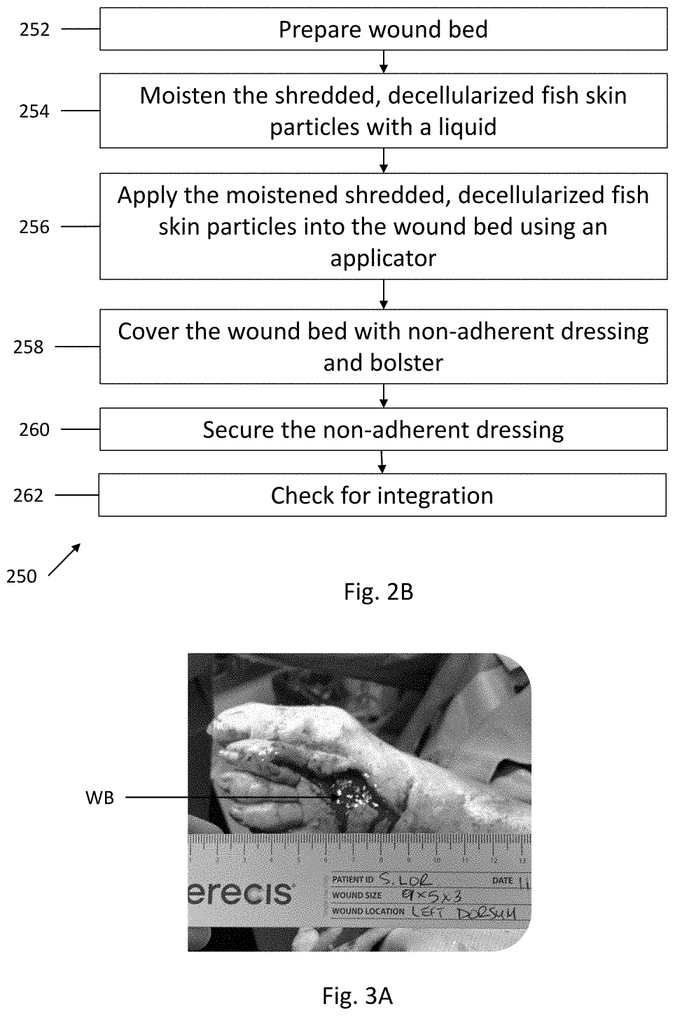

Turning to FIG. 2A, a method for wound treatment according to an embodiment of the present disclosure is shown and described. The method 200 corresponds generally to dry applications of the wound treatment and may include one or more of the following steps, not necessarily in the depicted order. The method 200 may comprise a step 202 of preparing a wound bed. The wound bed may be prepared, in embodiments, by cleaning the wound bed, such as by removing necrotic tissue and/or irrigating the wound bed to remove debris and exudates. In embodiments, the step 202 of preparing the wound bed includes removing previously applied wound treatments comprising shredded, decellularized fish skin particles, such as upon determining that the shredded, decellularized fish skin particles have not integrated after a predetermined threshold period. The predetermined threshold period may be 7 days, 10 days, 2 weeks, or any other suitable threshold.

The method 200 may further include a step 204 of applying dry shredded, decellularized fish skin particles from a container into the wound bed. The step 204 of applying the dry shredded, decellularized fish skin particles may include any suitable application method, such as pouring, sprinkling, packing, pressing, molding, combinations thereof, or otherwise.

For example, when performing the step 204 a clinician may pour a first layer of the dry shredded, decellularized fish skin particles directly onto the wound bed from a package and then, using an applicator such as gloved fingers, apply a pinch of the dry shredded, decellularized fish skin particles into a complex shape of the wound bed, such as at a tunneling or undermined wound. The clinician may apply enough of the dry shredded, decellularized fish skin particles to substantially or entirely fill a void defined by the wound bed, such as a tunneling wound. In embodiments, the step 204 may include pouring the dry shredded, decellularized fish skin particles into a clean or sterile container before applying the dry shredded, decellularized fish skin particles to the wound bed in any suitable manner.

The method 200 may further include a step 206 of covering the wound bed. The step 206 of covering the wound bed may include a covering utilizing or comprising a non-adherent dressing and optionally a bolster to ensure contact between the shredded, decellularized fish skin particles and the wound bed. That is, the step 206 of covering the wound bed may include arranging the covering, such as a non-adherent dressing, sufficiently flush against the wound bed so as to force the shredded, decellularized fish skin particles thereagainst. The non-adherent dressing may be any suitable dressing such as a synthetic non-woven non-adherent dressing, a cotton woven non-adherent dressing, or otherwise. The covering may include any suitable covering.

The step 206 may alternatively or additionally include securing the covering with foam or gauze to maintain moisture in the wound bed and to manage exudate. The method 200 may further include a step 208 of wrapping the wound bed to secure the covering.

The method 200 may further include a step 210 of checking for integration of the shredded, decellularized fish skin particles after a predetermined threshold. For example, the predetermined threshold may be a period of two weeks, which gives the particles sufficient time to integrate, i.e. to promote cellular ingrowth and neovascularization. If it is determined upon performing the step 210 that integration has not occurred, the method 200 may be repeated with the previously applied shredded, decellularized fish skin particles removed as part of the step 202 of preparing the wound bed.

Turning to FIG. 2B, a method for wound treatment according to an embodiment of the present disclosure is shown and described. The method 250 corresponds generally to wet applications of the wound treatment and may include one or more of the following steps, not necessarily in the depicted order. The method 250 may comprise a step 252 of preparing a wound bed as described above regarding the method 200.

The method 250 may further include a step 254 of moistening the shredded, decellularized fish skin particles with a liquid. The liquid may be any suitable liquid, such as saline of a predetermined concentration. In embodiments, the liquid may be 0.9% saline as is known to persons skilled in the art. The step 254 may include adding a predetermined quantity of the liquid, such as 1 cc, 2 cc, or otherwise.

The method 250 may include a step 256 of applying the moistened shredded, decellularized fish skin particles into or onto the wound bed using an applicator. The step 256 may involve forming the moistened shredded, decellularized fish skin particles into a paste before applying the paste to the wound bed using a gloved finger, a tongue depressor, a surgical tool, or any other suitable applicator. As with the method 250, applying the moistened shredded, decellularized fish skin particles may include applying enough of the particles to substantially or entirely fill a void defined by the wound bed, such as a tunneling wound. In other embodiments, the step 256 includes applying a layer of the moistened shredded, decellularized fish skin particles over the wound bed in addition or alternatively to filling a void.

The method 250 may further include steps 258 of covering the wound bed, with the moistened shredded, decellularized fish skin particles applied thereto, as described above regarding the step 206, 260 of securing the dressing as described above regarding the step 208, and/or a step 262 of checking for integration as described above regarding step 210 of the method 200.

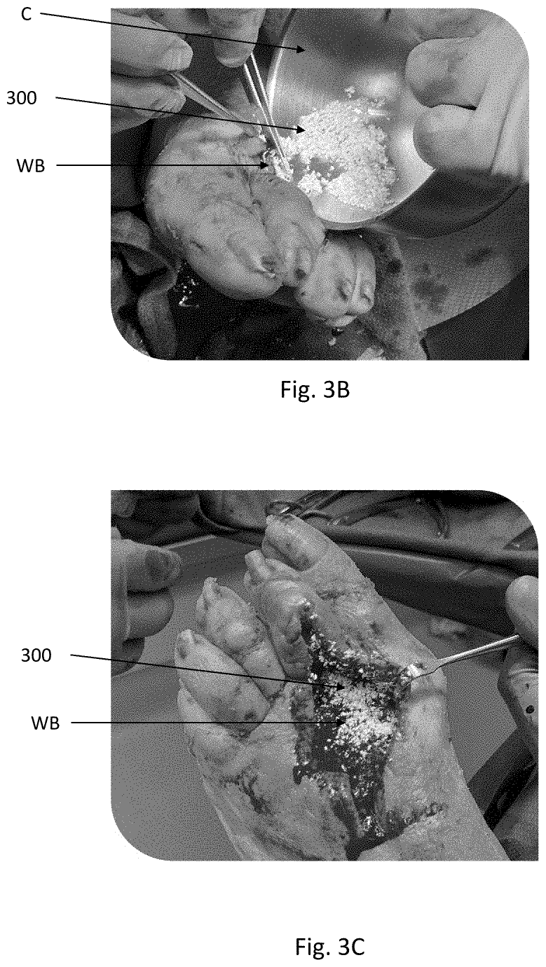

Turning to FIGS. 3A-3C, an application of a wound treatment according to an embodiment of the present disclosure is shown and described. FIG. 3A shows a wound bed WB that has been prepared for the wound treatment of the present disclosure. The wound bed WB of FIG. 3A is wound bed having one or more deep areas. As described above regarding FIGS. 2A and 2B, the wound bed WB may be prepared by first removing necrotic tissue and irrigating the wound bed WB to remove debris and exudates.

A wound treatment 300, comprising one or more dry shredded, decellularized fish skin particles (first removed from a packaging and added to a sterile container C), is then applied to the wound bed WB as shown in FIG. 3B. The wound treatment 300 may be poured, sprinkled, packed, or otherwise applied, such as using an applicator like a gloved finger, surgical tool, or otherwise. The wound treatment 300 may be added in sufficient quantities to substantially fill the one or more deep areas of the wound bed WB.

FIG. 3C shows the wound bed WB with the wound treatment 300 applied so as to substantially fill the one or more deep areas of the wound bed WB. The wound treatment 300 advantageously allows for the scaffold materials to fill the wound bed WB, which has a complex geometry, without extensive and time-consuming work by a clinician to cut a sheet-based decellularized fish skin material to size. Further, the wound treatment 300 advantageously fills the complex geometry, including the one or more deep areas, using a simple application procedure. By providing the wound treatment 300, the shredded, decellularized fish skin particles advantageously facilitate cellular ingrowth throughout the complex geometry of a wound while also providing the anti-viral and anti-bacterial benefits of Omega 3 PUFAs provided thereby.

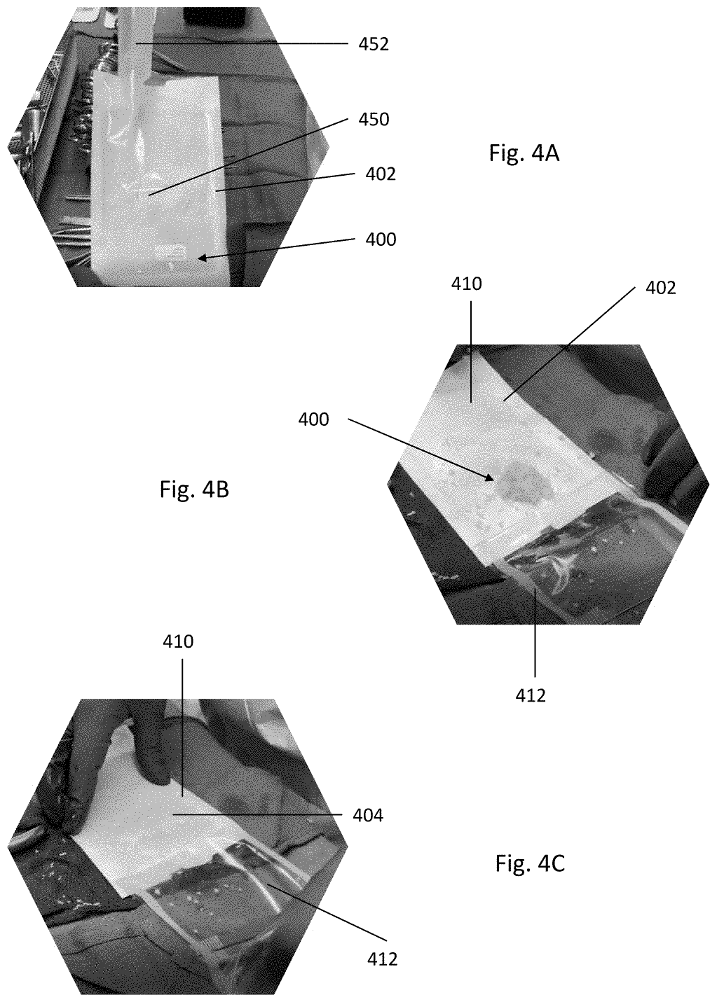

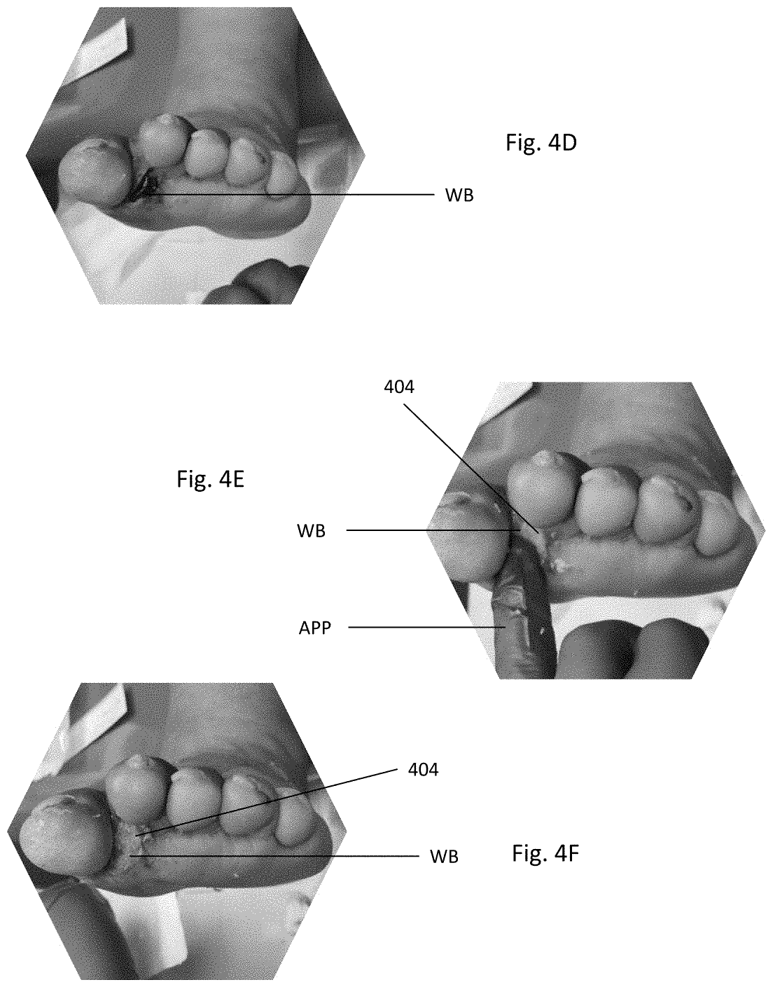

Turning now to FIGS. 4A-4F, an application of a wound treatment according to an embodiment of the present disclosure is shown and described. A wound treatment 400 is applied in a wet or moistened form to a wound bed as described regarding the method 250 of FIG. 2B. The wound treatment 400, comprising shredded, decellularized fish skin particles of a suitable size distribution as described above, may be provided in a package 402. The package 402 may be formed of any suitable material, such as a polymeric material, suitable for receiving a liquid 450 such as 0.9% saline, in an inner pocket defined by the package 402. The liquid 450 may be delivered using any suitable modality, such as a syringe 452 or other device. In embodiments, the syringe 452 may define indicia allowing the clinician to provide a predetermined amount of the liquid 450, such as 1 cc or 2 cc. The package 402 may be formed of a clear or see-through material so that a clinician may view the dry shredded, decellularized fish skin particles 400 as the liquid 450 is added thereto. Turning to FIG. 4B, the package 402 may be configured to be opened, such as by peeling a layer away, to allow the clinician to access the moistened fish skin particles 400 therein.

The moistened shredded, decellularized fish skin particles 400 may be formed, as shown in FIG. 4C, into a paste 404, using any suitable applicator, such as a gloved finger. This may be performed within or on the package 402, on a sterile surface, or in any other suitable location.

Turning to FIG. 4D, a wound bed WB for application of the wound treatment 400 is shown. The wound bed WB may define a complex geometry, such as a tunneling and/or undermined wound and/or be located such that sheet-based decellularized fish skin is inadequate and/or difficult to apply. As seen in FIG. 4E, the paste 404 is applied using an applicator APP to the wound bed WB. The applicator APP may be a gloved finger, a tongue depressor, a surgical tool, or otherwise. In embodiments, the application of the paste 404 includes pressing or packing the paste 404 into the wound bed WB so as to fill or substantially fill a void defined by the wound bed WB. Because of the size of the shredded, decellularized fish skin particles, the wound treatment 400 paste 404 is configured to contour closely to the geometry of the wound bed WB.

As seen in FIG. 4F, the wound bed WB may be filled, in embodiments up to a skin surface, with the paste 404. The application of moistened shredded, decellularized fish skin particles may advantageously aid in the formability, shape-keeping, pliability, and removability of the wound treatment 400. Whereas existing approaches to wound treatments tend to ooze out of place during application, the paste 404 has surprisingly been found to hold in place and retain its shape, thereby improving the effectiveness of the wound treatment. This further reduces waste of the wound treatment.

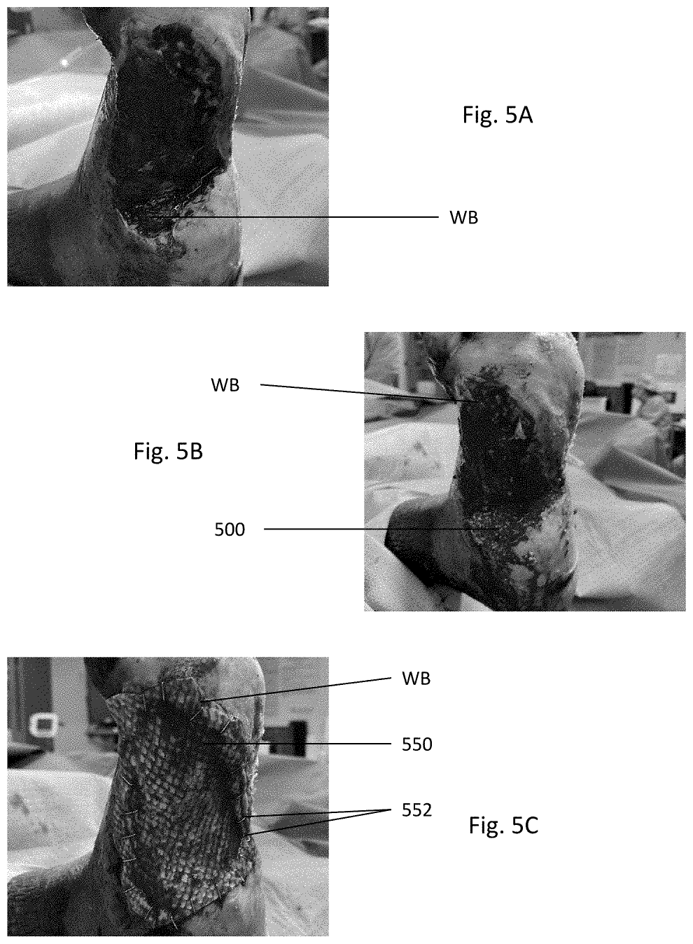

Turning to FIGS. 5A-5C, a wound bed WB is shown. The wound bed WB of FIG. 5A is a moderate to large-size wound with an uneven, deep area. The wound treatment 500 may be applied to the wound bed WB in dry or wet form and may be applied in a sufficient quantity to fill or substantially fill a desired portion of the wound bed. In the embodiment of FIGS. 5A-5C, a further step of applying a sheet-based decellularized fish skin material 550 is performed. The sheet-based decellularized fish skin material may be a scaffold as taught in U.S. Pat. No. 8,613,957. The sheet-based decellularized fish skin material 550 advantageously retains the wound treatment 500 in place while itself facilitating wound healing, cellular ingrowth, and neovascularization. One or more fasteners 552 hold the sheet-based decellularized fish skin material 550 in place relative to the wound bed WB.

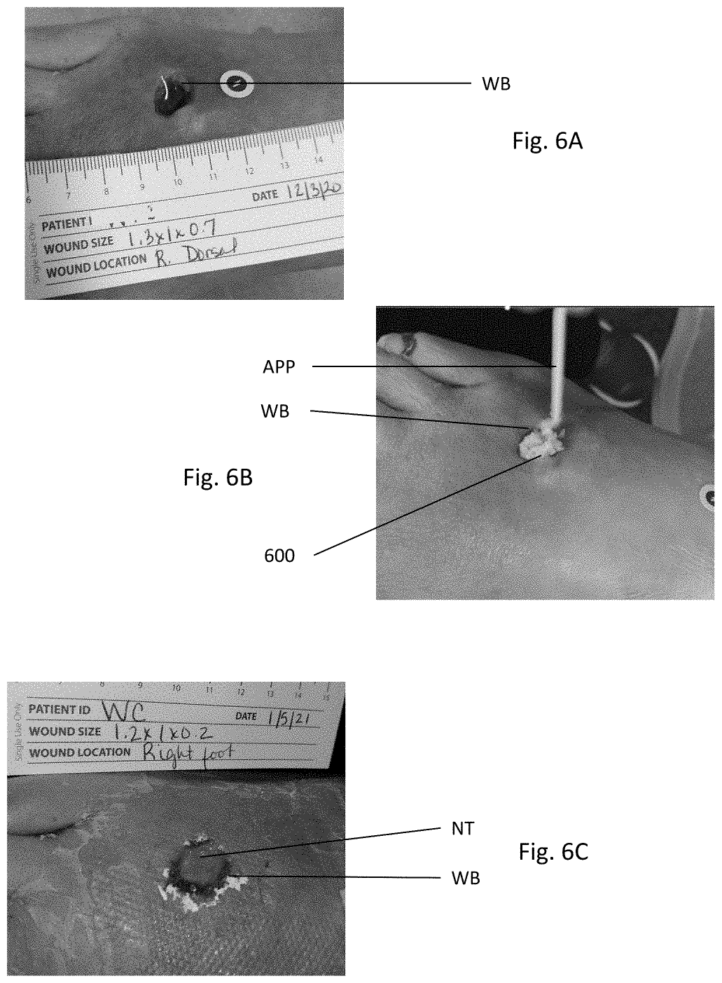

Turning to FIGS. 6A-6C, a wound bed WB is shown. The wound bed WB of FIG. 6A is a deep wound that does not lend itself well to existing sheet-based scaffold materials due to its depth. The wound treatment 600 according to embodiments of the present disclosure may be added to the wound bed WB using an applicator APP, such as a surgical tool, such that the wound treatment 600 fills or substantially fills the wound bed WB in its entirety. Because of the dimensions of the shredded, decellularized fish skin particles of the wound treatment 600, the wound treatment 600 may be easily, quickly, and effectively added to the wound bed WB with reduced waste, cost, and time while providing scaffold material within the wound bed WB to promote cellular ingrowth throughout a complex wound geometry. As seen in FIG. 6C, new tissue NT is ultimately yielded from the wound treatment 600.

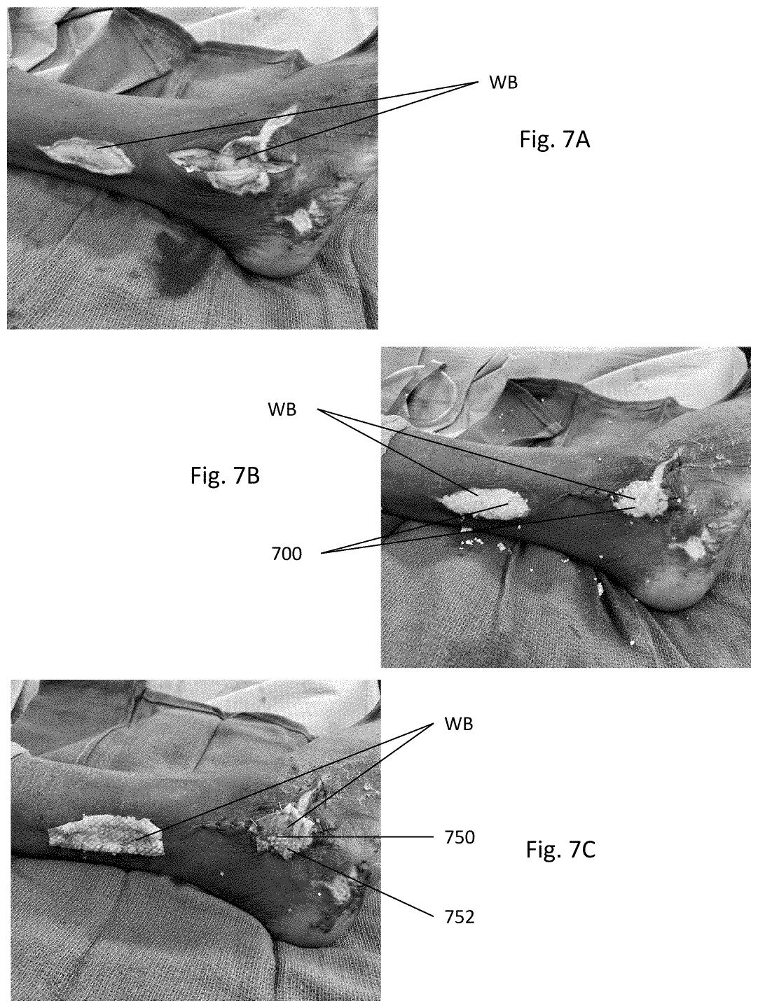

Turning to FIGS. 7A-7C, a wound bed WB with an irregular shape is shown. The WB comprises a plurality of wound beds of different shapes and depths, which complicates the application of existing wound treatments. As seen in FIG. 7B, a wound treatment 700 is applied as described herein to the plurality of wound beds WB in either dry or wet format such that the wound treatment 700 substantially conforms to the complex geometry of the wound bed WB. This can be done with minimal effort, time, and waste thanks to the dimensions of the shredded, decellularized fish skin particles, which despite their small size (which allows them to be packed into a wound bed) nevertheless provide scaffold material that promotes cellular ingrowth and neovascularization while also providing salubrious Omega 3 PUFAs to the wound bed WB.

As with the embodiment of FIGS. 5A-5C, a further step of applying a sheet-based decellularized fish skin material 750 is performed. The sheet-based decellularized fish skin material 750 may be a scaffold as taught in U.S. Pat. No. 8,613,957. The sheet-based decellularized fish skin material 750 advantageously retains the wound treatment 700 in place while itself facilitating wound healing, cellular ingrowth, and neovascularization. One or more fasteners 752 hold the sheet-based decellularized fish skin material 750 in place relative to the wound bed WB.

Temporary Wound Treatment for Stabilizing and/or Protecting a Traumatic Wound

In one or more other embodiments a temporary wound treatment comprising comminuted decellularized fish skin in particle form is provided. Preferably, the particle form of comminuted decellularized fish skin is configured to minimize cellular scaffolding at a wound site during temporary wound treatment.

This facilitates, in embodiments, a temporary wound treatment that stabilizes and/or protects a wound, for example preparatory to receiving subsequent or higher-level care. For example, the temporary wound treatment may be configured to protect and preserve a wound until a clinician removes the wound treatment for further treatment. By minimizing cellular scaffolding, the temporary wound treatment may be removed without exacerbating the wound or removing necessary cellular and vascular growth and structures.

In some embodiments, the temporary wound treatment additionally includes comprises a temporary bandage configured and arranged to deliver the comminuted decellularized fish skin in particle form to a wound. The temporary bandage can include, for example, a contact layer configured to interface with a wound and to retain the comminuted decellularized fish skin at a wound and an outer cover associated with the contact layer and configured to retain the contact layer at a wound.

Temporary wound treatments for stabilizing and/or protecting a wound can also include a base material, such as a biocompatible polymer, infused with or otherwise carrying comminuted decellularized fish skin particles. Additionally, or alternatively, temporary wound treatments for stabilizing and/or protecting a wound can include a compression element associated with the outer cover that is configured to conform the outer cover to the shape of the wound and/or to the shape of a partial or whole limb comprising the wound. The compression element can include, for example, a sleeve having an inflatable bladder. In some embodiments, the temporary wound treatment can include a base material disposed at a bottom or a peripheral wall of the sleeve and associated with the contact layer.

The comminuted decellularized fish skin particles within the temporary wound treatments can be smaller than about 1 cm in diameter, smaller than about 0.1 cm in diameter, smaller than about 10 mm in diameter, smaller than about 1 mm in diameter, smaller than about 0.1 mm in diameter, smaller than about 10 μm in diameter, smaller than about 1 μm in diameter, or combinations thereof. Additionally, or alternatively, the comminuted decellularized fish skin in particle form can be partially processed, such as by treating with enzyme(s) to reduce a rigidity of the comminuted decellularized fish skin particles. In some embodiments, such partial processing can cause at least a portion of extracellular matrix material within the partially processed, comminuted decellularized fish skin particles to be cleaved by the enzyme(s), increasing the ductility and/or elasticity of the partially processed, comminuted decellularized fish skin particles.

Embodiments of the present disclosure additionally include kits for stabilizing and/or protecting a wound. An exemplary kit can include a container including comminuted decellularized fish skin in particle form for placement on or in a wound and to be retained at a wound site by a contact element.

In some embodiments, the container further includes a contact element configured to interface with a wound and to retain the comminuted decellularized fish skin in particle form at a wound and an outer cover configured to retain the contact element and the comminuted decellularized fish skin in particle form at a wound. The container can additionally include a base material for carrying the comminuted decellularized fish skin in particle form and for associating with the contact element. The container may also include one or more therapeutics comprising analgesics, anesthetics, cytokines, growth factors, hemostatic agents, antibiotics, antifungals, hydrating compounds, or combinations thereof.

Embodiments of the present disclosure additionally extend to methods for stabilizing and/or protecting a wound. An exemplary method can include applying comminuted decellularized fish skin in particle form to a wound.

In some embodiments, methods for stabilizing and/or protecting a wound can include covering a wound with a contact element configured to retain the comminuted decellularized fish skin particles at a wound; and applying an outer cover to the contact element to secure the contact element to a partial or whole limb comprising a wound. In some embodiments, applying comminuted decellularized fish skin in particle form to a wound does not promote ingrowth of cellular tissue within the decellularized fish skin in particle form.

Embodiments of the present disclosure additionally extend to bandages for treatment of an unclean and/or non-debrided wound site. An exemplary bandage can include comminuted decellularized fish skin in particle form. The comminuted decellularized fish skin in particle form can be rehydrated prior to application to a wound site. The bandage can also include a covering to secure the comminuted decellularized fish skin in particle form at a wound site, such as a deep wound where it can be compacted into the deep wound.

Accordingly, temporary wound treatments, bandages, kits, and methods for stabilizing and/or protecting a wound are disclosed.

Current pre-hospital battlefield trauma care, including extremity hemorrhage, focuses solely on controlling bleeding until the wounded soldier can receive definitive care. Those treatments consist of application of pressure and tourniquets and wound covering with gauzes and hemostatic dressings. However, evacuation times for injured soldiers is expected to increase, and in some instances, soldiers are already positioned at remote locations where evacuation may take a number of days, amplifying the need for more advanced pre-evacuation treatments. In addition to controlling hemorrhage, pre-evacuation treatments are need that prevent infections, minimize further tissue loss, protect underlying tissues/organs, reduce ischemia and secondary injury, and reduce pain and suffering.

Preservation of tissue conditions is important for minimizing the loss of damaged tissue (including tissue surrounding the wound) caused by necrosis, debridement, or amputation and is also important for maximizing the likelihood that the affected area can be rehabilitated. If a traumatic injury can be promptly and properly treated, there is a higher likelihood that the affected area can be rehabilitated while minimizing the loss of structure and/or function at or surrounding the wound site. However, the medical facilities and equipment necessary to properly treat traumatic wounds and to maximize positive outcomes from rehabilitation are often available at permanent hospitals or possibly within centralized field hospitals.

When traumatic wounds are experienced at remote locations, such as on the battlefield, it is difficult to preserve the damaged tissue for the extended periods of time necessary to transport the injured individual to appropriate treatment facilities. In some instances, it could take days to transport an injured soldier to an appropriate treatment facility. As a result, traumatic wounds received in remote locations are associated with a high degree of tissue necrosis, require a larger area of tissue debridement prior to treatment, and are more likely to lead to amputation and poorer rehabilitation outcomes.

Embodiments of the present disclosure enable and improve prolonged field treatment of wounds, particularly traumatic wounds, burns, and/or amputations and allow for the stabilization and/or preservation of damaged tissue. Embodiments of the present disclosure include treatments and kits related thereto that decrease the loss of damaged tissue and/or increase the likelihood that a wound can be successfully rehabilitated. This is enabled, at least in part, from the incorporation of a particle form of comminuted decellularized fish skin at the wound site. Once applied to the wound site, particle forms of the comminuted decellularized fish skin act to stabilize and/or preserve the wound site. The particles can be applied to an unclean wound and/or non-debrided wound on site and can beneficially act to reduce infections, reduce pain associated with the wound, and/or reduce a need for repeated wound debridement. Embodiments disclosed herein can be used to prepare wounds for effective autografting or skin flap closure, which increases the likelihood of autograft-take.

Advantageously, the comminuted decellularized fish skin particles are configured in embodiments to minimize cellular scaffolding such that the temporary wound treatment may be removed during subsequent treatment without damaging newly grown cellular structures.

As used herein, the term “treatment” is intended to be understood by its common dictionary definition. That is, the term “treatment” broadly includes medical care and/or medicaments given to a patient for an illness or injury. As should be appreciated by those having skill in the art, a “treatment” includes the use of a chemical, physical, or biological agent to preserve or give particular properties to something. Thus, a “treatment” may be the medical care provided (i.e., in the form of a method or series of prescribed acts), or it may refer to the medicament used to preserve or give a particular property to something.

As a non-limiting example, the particle form of decellularized fish skin disclosed herein can be referred to as a “treatment”—i.e., a medicament used to preserve and/or stabilize a wound or which can provide any of the other disclosed beneficial effects to a wound site. Similarly, in some instances, a treatment includes use of the disclosed decellularized fish skin in particle form within methods for stabilizing and/or protecting a wound.

As stated above, it is the natural three-dimensional structure of extracellular matrix components acting as a scaffold for cell infiltration and outgrowth that is fundamental to promoting wound healing. To act as a scaffold, however, the wound site is usually debrided and cleaned before the decellularized fish skin is applied. Removal of dead or damaged skin allows direct access to the sub-epidermal tissues upon which the new skin layer can form. It, therefore, would be counterintuitive and seemingly undesirable to use intact decellularized fish skin in conditions or dimensions unfavorable to cellular ingrowth or, further, to disrupt the three-dimensional structure of natural extracellular matrix components.

Surprisingly, however, the mechanical comminution of decellularized fish skin, as disclosed herein, and its subsequent use in particle form on dirty, non-debrided wounds results in some unforeseeable advantages. Many of the salubrious properties of decellularized fish skin in particle form are independent from the tissue-repair properties of decellularized fish skin when used as a scaffolding material, and the particle form of comminuted decellularized fish skin can be advantageously utilized to preserve tissue while not substantially repairing it.

For example, Applicant discovered that application of comminuted decellularized fish skin particles at a wound site according to an embodiment of the present disclosure, with or without one or more other elements disclosed herein, provides the unforeseeable effect of stabilizing and/or protecting the wound and surrounding tissue instead of actively promoting wound healing as a physical scaffold for infiltrating cells involved in wound healing/repair.

These surprising and unexpected wound preservation and stabilization properties allow the comminuted decellularized fish skin particles of embodiments to be utilized in new and previously unforeseen scenarios and, in some embodiments, may be used to serve a different purpose than that previously envisioned for decellularized fish skin scaffolds. For example, comminuted decellularized fish skin in particle form can be used as part of a temporary bandage where the decellularized fish skin particles act to stabilize the wound until further medical treatment can be administered.

In some embodiments, the comminuted decellularized fish skin particles are applied directly to a dirty (e.g., uncleaned or non-debrided) wound to stabilize and/or protect the wound until the wound can be cleaned, debrided, and treated at a properly equipped and staffed medical facility. The comminuted decellularized fish skin in particle form is not intended to promote wound healing, particularly as a scaffold material. Instead, the comminuted decellularized fish skin particles are provided at a wound site and later removed during cleaning/debriding of the wound prior to subsequent treatment. As described herein, the comminuted decellularized fish skin particles can beneficially provide increased hemostatic properties to the wound to manage/reduce blood loss, help maintain a proper moisture environment at the wound site to prevent desiccation of further damage to the tissue, provide antibacterial and antiviral defense, regulate inflammation, and in some instances, reduce pain.

This is in contrast to other applications and embodiments of decellularized fish skin where it is used primarily, and is particularly advantageous, as a scaffold material that promotes cellular ingrowth for long-term wound healing treatments. If decellularized fish skin scaffolding was applied to a cleaned and debrided wound but removed during the wound healing process (e.g., days later), the wound would be at least partially reopened as the scaffold material—and all the wound-healing cells intercalated/associated therewith—is ripped away from the wound site. Such action is likely to cause additional trauma to the wound site and exacerbate the problem instead of stabilizing and/or protecting it.

On the other hand, comminution of the decellularized fish skin into particle form according to embodiments transforms the decellularized fish skin scaffold and enables additional treatment benefits and options, including, for example, use on dirty and/or non-debrided wounds as a hemostatic agent that temporarily preserves the wound site and allows for extraction of the wounded individual to a distant medical care facility where the wound can be subsequently treated. The comminuted decellularized fish skin particles can be used easily in the field without the need for any special medical training. Comminuted decellularized fish skin particles can be included, with or without a substrate or even as part of a temporary bandage, to stabilize and/or protect wounds. This can allow for improved and/or prolonged field care treatments, which among other things increases the likelihood of rehabilitating the affected area.

Additionally, comminution allows the decellularized fish skin to be applied more dynamically and more quickly to wound sites than non-comminuted forms. In some embodiments, this allows some salubrious properties of decellularized fish skin, such as its hemostatic properties, barrier function properties, and/or pain relieving properties, to figure more prominently in treatments.

As used herein, the term “comminution” refers to the action of reducing a material to smaller fragments or particles. Comminution may occur by any process or force, including without limitation, mechanical force (e.g., cutting, shredding, tearing, crushing, shearing, grinding, jet milling, etc.), focused heat (e.g., laser cutting), any other process or mechanism for reducing material to fragments or particles, or any combinations of the foregoing. For example, very sharp blades and/or a low RPM mill can be used to comminute decellularized fish sheets into particulate form.

Accordingly, the term “comminuted” may be used herein to refer to a physical property of a material and/or a resultant product, one that has undergone comminution. Thus, the term “comminuted decellularized fish skin,” as used herein, may denote a physical property of the decellularized fish skin—that the decellularized fish skin is represented as smaller fragments or particles—or it may additionally, or alternatively, denote a resultant product—decellularized fish skin that has been reduced to fragments or particles. It should be appreciated that in some embodiments, comminution reduces the physical size of the decellularized fish skin in at least one dimension, but it may not affect the ultrastructure of the particles. That is, some embodiments of comminuted decellularized fish skin are physically reduced in size but retain their three-dimensional extracellular structure.

The varying forms of comminuted particles can be separated using a sieve or hole filter or otherwise selected based on the method of comminution.

In addition to the foregoing and as described briefly within the background, soldiers deployed in battle zones are at high risk of injury from blast wounds. In future conflicts, it is likely that extraction of injured soldiers will become increasingly difficult due to fighting in remote locations, urban environments, and other circumstances where there is diminished communication and air superiority. This change in the combat environment requires alternative solutions to treat field injuries, as current standards of care may not be practical.

Similarly, wounds received in a rural setting (e.g., resulting from use of heavy machinery, hunting accidents, injuries related to an all-terrain vehicle crash or roll-over, or similar) can benefit from alternative solutions to treat injuries sustained in an environment where a hospital or professional healthcare setting is difficult or time-consuming to reach.

In particular, wound care solutions are needed that are rugged and robust (e.g., long half-life, stability at extreme temperatures and conditions), lightweight, small, easy to transport, simple and quick to handle at the point of injury, have little to no dependency on external power or specialized equipment, and modular and interoperable (e.g., can be integrated with current approaches to care).

Embodiments of the present disclosure solve one or more of the foregoing problems and can exhibit many, if not all, of the aforementioned desired properties. For example, kits including a source of comminuted decellularized fish skin particles can be used in the field for the preservation and/or stabilization of wounds (e.g., combat-related traumatic wounds).

For example, when packed into asymmetrical deep-tissue wounds in injured soldiers, comminuted decellularized fish skin has an application as a hemostatic agent, antibacterial agent, and can kick start dermal regeneration. This can be achieved in the field and/or at field-based medical facilities close to the point of injury instead of at state-of-the-art healthcare facilities where similar benefits have traditionally been realized.

In some embodiments, sheets of decellularized fish skin can be used either in combination with comminuted decellularized fish skin or alone to provide immediate hemostatic and antibacterial properties for covering wound sites. When used in combination, sheet-based and comminuted decellularized fish skin can bring benefits not realized with use of sheet-based products alone, such as enhanced wound bed granulation, making the wound more suitable for early grafting or flap surgery, and/or ultimately providing a better functional and aesthetic outcome, as well as providing long-term benefits such as increased wound healing rates.

It should be readily appreciated that embodiments disclosed herein can also translate directly to public use (e.g., in shock and trauma centers), especially in rural areas where sub-optimal health care and/or infrastructure may prevent the ideal treatment of traumatic wounds, such as deep-tissue wounds.

### Exemplary Embodiments

Embodiments of the present disclosure provide field-ready devices, kits, and compositions, for stabilizing, covering, and/or initializing tissue regeneration of deep-tissue wounds for prolonged and en route care.

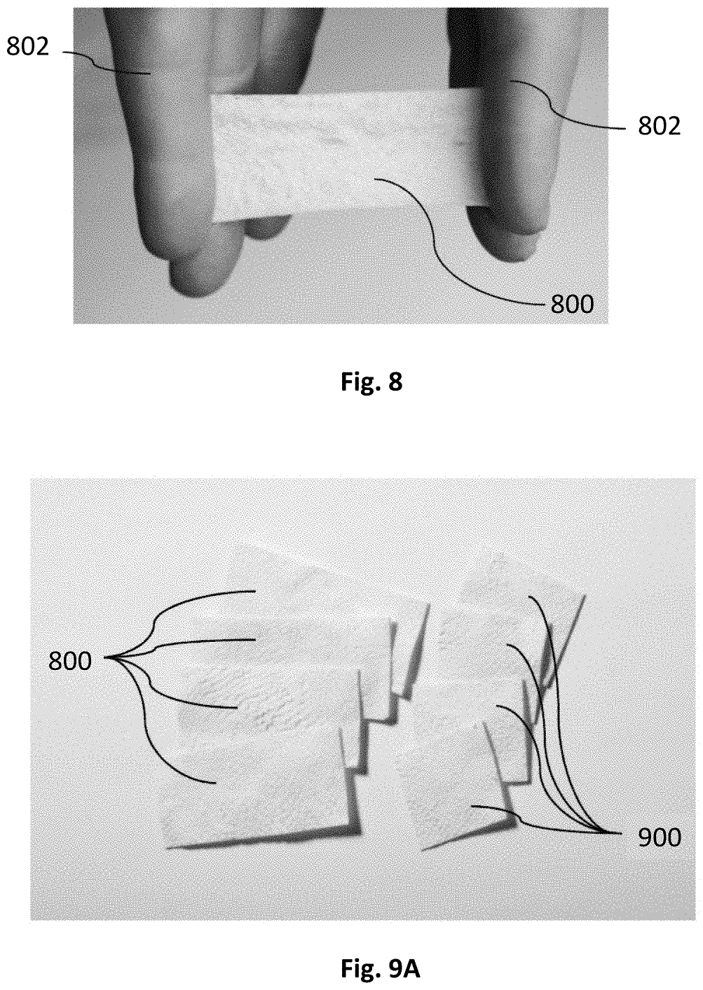

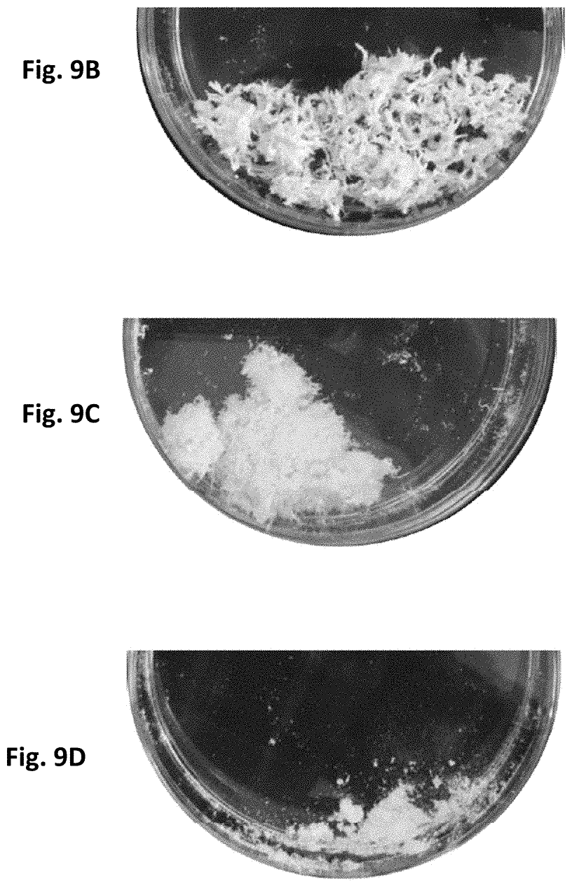

Referring now to FIG. 8 and FIG. 9A, illustrated are exemplary embodiments of decellularized fish skin 800, 900 before comminution and/or other treatments. An exemplary section of decellularized fish skin 800, made as described in U.S. Pat. No. 8,613,957, is illustrated in FIG. 8 with the size thereof given context by the user's gloved hands 802.

It should be appreciated that the decellularized fish skin can be comminuted or processed into various sizes. As shown in FIG. 2A, a plurality of decellularized fish skin sheets 800, 900 can be sized and shaped similar to the decellularized fish skin 800 of FIG. 8 (e.g., rectangular) or they can have more uniform dimensions (e.g., squares), such as the decellularized fish skin sheets 900 illustrated in FIG. 9A.

The decellularized fish skin scaffold 800, 900 depicted in FIGS. 8 and 9A is substantially rigid and inelastic in lyophilized form. The decellularized fish skin scaffold can be treated with one or more enzymes that act to increase its ductility and/or elasticity. In some embodiments, the enzymes act by cleaving interconnected extracellular matrix components without substantially impacting the salubrious properties important for wound preservation and/or stabilization. In some embodiments, the enzymes cleave covalent bonds within and/or between elastins, proteoglycans, collagens, or other extracellular matrix materials, but the modified decellularized fish skin retains a substantial portion of the extracellular matrix contents, even if partially removed from its natural three-dimensional structure.

In some embodiments, the enzyme treatment negatively impacts the use of the modified decellularized fish skin as a scaffold material. It should be appreciated, however, that loss of function as a scaffold material, surprisingly, does not appreciably impact the use of decellularized fish skin as a wound preservation and stabilization material. Thus, the ductility and/or elasticity of the material may be increased while maintaining the composition of the extracellular components, and even though this may negatively affect the use of the material as a scaffold for wound healing, the modified decellularized fish skin can nonetheless act as a wound preservation/stabilization material.

The decellularized fish skin scaffold can be comminuted and provided in particle form. It should be appreciated that the size of individual comminuted particles may vary, depending on the type and/or manner of comminution. For example, decellularized fish skin particles can be created through a jet milling process designed to output particles below a specified size. In some embodiments, decellularized fish skin is cut, chopped, or ground into particles, which may be done in a measured fashion to create uniform particles or roughly performed, thereby generating a variety of different sized particles.

In one embodiment, comminution of decellularized fish skin occurs by mechanically cutting sheets of decellularized fish skin into fragments and/or particles less than 1 cm in diameter. Alternatively, or additionally, comminution of decellularized fish skin occurs using a mechanical grinder, such as a hemp grinder.

The diameter of fragments and/or particles resulting from comminution may be less than about 0.1 cm, less than about 10 mm, less than about 1 mm, less than about 0.1 mm, less than about 10 μm, less than about 1 μm, or combinations thereof. That is, in some embodiments the diameter of fragments and/or particles may vary, being defined as a range of sizes between any combinations of the foregoing dimensions. In some embodiments, the particles may be size separated.

In some embodiments, the size of particles can be uniform or within a range of uniform or varying sizes. For example, comminuted decellularized fish skin particles can be size selected through one or more sieves or screens, which retain bulky particles and allow particles smaller than the sieve mesh size to pass through. The particles passing through the sieve are within a range of sizes, having an upper threshold at the sieve mesh size of the first sieve. In some embodiments, additional (or a series of) size selection sieve(s) can be used to further refine the particle size.

For example, a sharp-bladed mill processing at a low RPM can be used to comminute decellularized fish skin. The mill can be associated with hole filters to allow for a relatively reliable size-selection of product. In such an embodiment, scale up could be facilitated by a controlled temperature, low-RPM mill.

Continuing with the foregoing example of decellularized fish skin particles that have been passed through a first sieve—and which are now defined by an upper threshold size—can be filtered over or through a sieve having a smaller mesh size. The particles that are undersized (e.g., pass through the second sieve) have an upper threshold particle size, which is smaller than the upper threshold size of the particles retained by the second sieve. The particles that are trapped by the second sieve are now additionally defined by a lower threshold particle size.

Different particle sizes may offer different benefits and may be chosen for different applications based on particle size. Additionally, or alternatively, a combination of different particle sizes may be selected for the combined effects offered by the various particle sizes. In some embodiments, particles can have an upper and/or lower threshold defined by passage through and/or retention by one or more sieves having openings sized to 1 cm, 10 mm, 8 mm (2½ Mesh), 6.73 mm (3 Mesh), 5.66 mm (3½ Mesh), 4.75 mm (4 Mesh), 4.00 mm (5 Mesh), 3.36 mm (6 Mesh), 2.83 mm (7 Mesh), 2.38 mm (8 Mesh), 2.00 mm (9 Mesh), 1.68 mm (10 Mesh), 1.41 mm (12 Mesh), 1.19 mm (14 Mesh), 1.00 mm (16 Mesh), 0.841 mm (20 Mesh), 0.707 mm (24 Mesh), 0.595 mm (28 Mesh), 0.500 mm (32 Mesh), 0.420 mm (35 Mesh), 0.354 mm (42 Mesh), 0.297 mm (48 Mesh), 0.250 mm (60 Mesh), 0.210 mm (65 Mesh), 0.177 mm (80 Mesh), 0.149 mm (100 Mesh), 0.125 mm (115 Mesh), 0.105 mm (150 Mesh), 0.088 mm (170 Mesh), 0.074 mm (200 Mesh), 0.063 mm (250 Mesh), 0.053 mm (270 Mesh), 0.044 mm (325 Mesh), 0.037 (400 Mesh), 0.025 mm (500 Mesh), an opening size larger than the foregoing (e.g., larger than 1 cm), an opening size between any of the foregoing (e.g., any size between 0.025 mm and 10 cm), or an opening size smaller than the foregoing (e.g., less than 0.025 mm).

Accordingly, following comminution, the comminuted decellularized fish skin may be: the same and/or different shapes, the same and/or different thickness (and/or the same and/or different width and/or length), pellet-shaped, flakes, powder (or powder-like), suspended as a colloid in a mixture/solution, combinations thereof, and/or any other physical state. In one embodiment, the comminuted decellularized fish skin is equilibrated in a solution before being applied to a wound and/or used in a temporary bandage. Examples of solutions include saline, water, alcohol, an antibiotic solution, a hydrating compound, and/or any aqueous solution with or without one or more therapeutics added (or dissolved) therein, and in some embodiments, the solutions may be sterile.

In one embodiment, the comminuted decellularized fish skin particles are part of a temporary bandage which are applied to/with and/or infused on a surface layer and/or within a contact layer or base material at a wound. It should be appreciated for the purposes of this disclosure that, where appropriate, embodiments disclosing the use of comminuted fish skin particles may additionally, or alternatively, incorporate or use decellularized fish skin or modified decellularized fish skin (e.g., enzymatically treated decellularized fish skin having increased ductility/elasticity). Additionally, or alternatively, the comminuted decellularized fish skin particles may be administered to the wound as a separate or distinct treatment before application of the temporary bandage (e.g., by sprinkling onto or coating the wound).

FIG. 9B illustrates an exemplary depiction of large particles of comminuted decellularized fish skin resulting from grinding a sheet of decellularized fish skin scaffold material with a hemp grinder. FIG. 9C illustrates an exemplary depiction of threaded, cotton-like fibers of comminuted decellularized fish skin resulting from grinding a sheet of decellularized fish skin scaffold material with a hemp grinder in accordance with embodiments of the present disclosure. FIG. 9D is an exemplary depiction of small, powder-like particles of comminuted decellularized fish skin resulting from grinding a sheet of decellularized fish skin scaffold material with a hemp grinder.

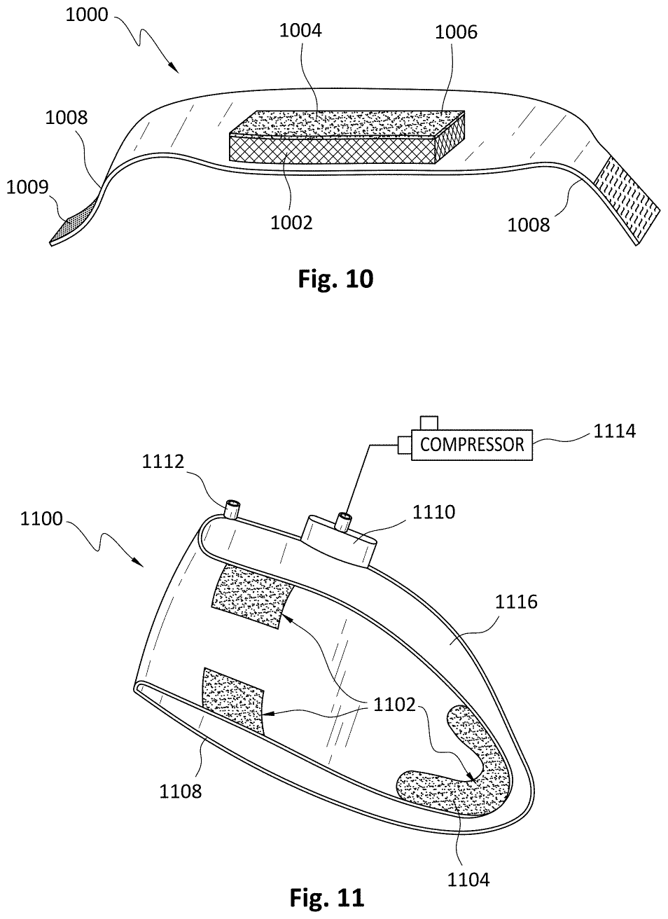

Referring now to FIG. 10, illustrated is a temporary bandage 1000. The temporary bandage 1000 is configured and arranged to deliver comminuted decellularized fish skin particles 1004 to a wound. The comminuted decellularized fish skin particles 1004 can be delivered and/or retained at a wound by an interior surface of the temporary bandage 1000 or by a contact layer 1006. The contact layer 1006 can include any contact layer material known in the art including, without limitation, a nonadherent material consisting of a perforated or woven polymer material, hydrogel, foam (e.g., polyurethane foam dressings), silicone, a porous material that is permeable to fluid (e.g., a hydrophobic silicone containing a plurality of apertures), combinations thereof, or any other suitable contact layer material. The contact layer 1006 can be permeable to fluids originating from the wound (e.g., blood, exudate, etc.) and may additionally be permeable to gases, allowing at least some circulation of air at the wound.

In some instances, the contact layer 1006 may be an impermeable barrier that primarily acts to deliver and/or retain the comminuted decellularized fish skin particles 1004 at the wound. Accordingly, the contact layer 1006 can include or be configured to retain the comminuted decellularized fish skin 1004 at a wound by, for example, acting as a transport and/or as a physical barrier to hold the comminuted decellularized fish skin particles 1004 at or proximate the wound site.

The contact layer 1006 can additionally, or alternatively, be configured to deliver the comminuted decellularized fish skin particles 1004 to the wound through hydrophobic channels defined thereby and/or therein. Such hydrophobic channels can resist absorption of fluids originating from the wound site but provide a medium through which such fluids can be drawn away from the wound.

Comminuted decellularized fish skin particles 1004 can be associated with the corresponding lumens of the hydrophobic channels in a lyophilized form where they do not stick or otherwise adhere to the channels, and upon wound exudate, blood, or other fluid originating from the wound site being drawn into the channels, the comminuted decellularized fish skin particles 1004 can be hydrated and diffuse to the wound surface to provide the benefits described above (e.g., hemostasis, analgesic effects, antimicrobial effects, barrier functions, etc.). In some instances, the comminuted decellularized fish skin particles 1004 can be applied to the wound directly (in a dried or hydrated form) in addition to being transported and retained at the wound site by the contact layer 1006.

In some instances, the contact layer 1006 can be or include a hydrogel, such as a hydrogel known to persons skilled in the art. Comminuted decellularized fish skin particles 1004 can be associated with a surface of the hydrogel such that the hydrogel acts to deliver and retain the comminuted decellularized fish skin particles 1004 at the wound site. Additionally, or alternatively, the comminuted decellularized fish skin particles 1004 can be incorporated into the hydrogel and released at the wound site. For example, a hydrogel including comminuted decellularized fish skin particles 1004 can be applied to a wound site, and upon absorbing water from the wound site, comminuted decellularized fish skin particles 1004 associated therewith can be released to diffuse to the wound site. Hydrogels may be additionally beneficial, as they can allow the contact layer 1006 to conform to the wound site as the hydrogel swells.

The temporary bandage 1000 can optionally include a base material 1002 associated with or comprising the contact layer 1006 and configured to interface with a wound and/or promote contact of the decellularized fish skin particles 1004 with the wound. The temporary bandage 1000 further includes an outer cover 1008 associated with and/or at least partially surrounding the contact layer 1006 and which is configured to retain the contact layer 1006 at the wound. As depicted in FIG. 10, the outer cover 1008 includes straps 1009 at opposing sides of the outer cover 1008. In one or more embodiments, the straps 1009 of the outer cover 1008 may be wrapped around, adhered to, and/or otherwise associated with a wound site and may be adhered thereto by any means known in the art (e.g., hook and loop on opposing ends of straps; adhesive material such as tape, glue, epoxy, cement, or similar applied to one or more straps and attached at or near the wound site or to an opposing strap or other part of the outer cover; tie/fuse straps together; etc.).

When included, a base material 1002 can include or be made of the same material as the contact layer 1006 described above or any other material, such as materials that promote a wound healing effect by a complex function of the release of therapeutics and/or the formation of a proper moisture environment by interfering with the influx of foreign substances and/or releasing and/or storing an exudate at a wound site to maintain a proper moisture environment. Such base materials include, for example, gels, semi-solids, biocompatible polymers, and/or any combination thereof. In some embodiments, the base material 1002, outer cover 1008, and/or straps 1009 are stretchable or moldable, being configured to conform to the wound site and/or firmly compress the wound site.

In some embodiments, a base material 1002 includes a biocompatible polymer. Any polymer materials usable as a dressing material may be used as the biocompatible polymer without limitation and may be properly chosen by those skilled in the art. The biocompatible polymer may, for example, include one or more of: polyvinyl alcohol, polyurethane, polyethylene, polyethylene oxide, low-density polyethylene, polyacrylic acid, polyoxyethylene, polytetrafluoroethylene, polypropylene, polyethylene terephthalate, polyamide, polyacrylonitrile, polyester, polyvinyl chloride, polyvinylidenefluoride, polysiloxane (a silicone rubber), polyglycolic acid, polylactic acid, polymethacrylic acid, polyacrylamide, polysaccharide, polyvinylpyrrolidone, silicone, alginic acid, sodium alginate, cellulose, pectin, chitin, chitosan, gelatin, collagen, fibrin, hyaluronic acid, natural rubber, synthetic rubber, or combinations thereof.

The biocompatible polymer may be prepared by gathering the biocompatible polymer in a fibrous form and processing the biocompatible polymer into a sheet or sheet-like shape, or fibrous biocompatible polymers may be processed into a non-woven fabric or a woven fabric. Additionally, or alternatively, the biocompatible polymer may be used in the form of a film, foam, hydrocolloid, hydrogel or may be properly processed in any other form as known by those skilled in the art.

In one embodiment, the base material 1002 and/or biocompatible polymer helps, at least in part, to regulate moisture content of a wound, as known in the art or as otherwise described herein.

In one or more embodiments, the temporary bandage 1000 includes one or more therapeutics, which may, in some embodiments, include one or more of analgesics, anesthetics, cytokines, growth factors, hemostatic agents, antibiotics, antifungals, hydrating compounds, and combinations thereof. The therapeutics and/or the comminuted decellularized fish skin may be infused within the base material 1002 (e.g., within a biocompatible polymer) for time and/or temperature dependent release as known in the art (e.g., hydrogels).

In one or more embodiments, analgesics include: acetaminophen, non-steroidal anti-inflammatory drugs (NSAIDs), opioids, and the like. Analgesics may additionally, or alternatively, include drugs for treating neuropathic pain such as, for example, tricyclic antidepressants and anticonvulsants.

In one or more embodiments, anesthetics include ester- and/or amide-based local anesthetics. Ester-based local anesthetics include, for example, procaine, amethocaine, cocaine, benzocaine, tetracaine, and the like. Amide-based local anesthetics include, for example, lidocaine, prilocaine, bupivicaine, levobupivacaine, ropivacaine, mepivacaine, dibucaine, etidocaine, and the like. In one embodiment, amide-based local anesthetics are preferred due to their heat-stability and longevity (e.g., shelf life of about two years).

In one or more embodiments, cytokines include pro-inflammatory cytokines, anti-inflammatory cytokines, and/or combinations of the pro- and anti-inflammatory cytokines. Anti-inflammatory cytokines include a series of immunoregulatory molecules that control the pro-inflammatory cytokine response and which may act in concert with specific cytokine inhibitors and soluble cytokine receptors to regulate the human immune response. In an embodiment, one or more anti-inflammatory cytokines are used to reduce pain, swelling, and other symptoms of inflammation at the wound site. Exemplary anti-inflammatory cytokines include, for example, interleukin (IL)-1 receptor antagonist, IL-4, IL-6, IL-10, IL-11, and TGF-β.

In one or more embodiments, growth factors include transforming growth factor alpha (TGF-α), vascular endothelial growth factor (VEGF), fibroblast growth factor (FGF), epidermal growth factor (EGF), matrix metalloproteinase 2 (MMP-2), MMP-9, and the like.

In one or more embodiments, antibiotics include one or more antibiotics selected from antibiotic classes of:
penicillins (e.g., methicillin, amoxicillin, penicillin G, etc.);
tetracyclines (e.g., doxycycline, tetracycline, etc.);
cephalosporins (e.g., cefdinir, cefepime, ceftriaxone, etc.);
quinolones/fluoroquinolones (e.g., ciprofloxacin, levofloxacin, ofloxacin, etc.);
macrolides (e.g., azithromycin, erythromycin, etc.);
sulfonamides (e.g., sulfisoxazole, sulfamethoxazole-trimethoprim, etc.);
glycopeptides (e.g., vancomycin, dalbavancin, etc.);
aminoglycosides (e.g., gentamycin, kanamycin, etc.);
lincosamides (e.g., clindamycin, lincomycin, etc.);
nitrofurans (e.g., furazolidone, nitrofurantoin, etc.);
oxazolidinones (e.g., linezolid, torezolid, etc.);
ansamycins (e.g., geldanamycin, rifaximin, etc.);
carbapenams (e.g., meropenem, erapenem, etc.); and
polypeptides (e.g., bacitracin, polymyxin B, etc.).

In an embodiment, the antibiotics include common topical antibiotic cocktails (e.g., bacitracin, neomycin/polymyxin B, neomycin/polymyxin/pramoxine, etc.). The antibiotic may be chosen from any known antibiotic not mentioned above, including, without limitation, fosfomycin, mupirocin, and chloramphenicol.

In one or more embodiments, antifungals include polyene antifungals (e.g., nystatin, amphotericin B, etc.), echinocandins (e.g., micafungin, caspofungin, etc.), azole antifungals (e.g., imidazoles such as, for example, bifonazole, sulconazole, and others; triazoles such as, for example, epoxiconazole, fluconazole, and others; and thiazoles such as, for example, abafungin), allylamines (e.g., butenafine, naftifine, etc.), and combinations thereof.

In one or more embodiments, hydrating compounds include materials that wick and/or store wound exudate and also include hydrating gels, oils, or other fluids known in the art. Non-limiting examples include petroleum jelly, beeswax, panthenol, and the like.

Referring now to FIG. 11, a temporary bandage may be associated with an outer cover 1108 in the form of a sleeve 1100 that includes, optionally, a biocompatible polymer 1102 infused with or otherwise associated with comminuted decellularized fish skin particles 1104 disposed at the base of the sleeve 1100. The biocompatible polymer 1102 may be ergonomically shaped and/or be deformable to receive a wound and/or partial or whole limb. The sleeve 1100 may additionally be associated with a compression device 1110. As depicted in FIG. 11, the compression device 1110 inflates the outer cover 1108 such that the inflated sleeve 1100 conforms to the wound and/or limb or partial limb within the sleeve 1100.

As shown in FIG. 11, the outer cover 1108 is inflated by filling an inner bladder 1116 with, for example, air from a compressor 1114. Alternatively, the compression device can be filled via a user's mouth, or it can be a manual pump. The inner bladder 1116 may alternatively be filled with a fluid (e.g., water).

The sleeve 1100, particularly the inner bladder 1116 of the sleeve 1100, can be vented or the pressure of the inner bladder 1116 adjusted by venting air or fluid filing the bladder 1116 through a vent 1112. In some instances, the vent 1112 is manually operated, though it may in some instances have a threshold pressure at which it automatically vents to maintain pressure within the inner bladder 1116 beneath a given threshold (e.g., 20 psi).

In one embodiment a sleeve additionally includes: one or more straps (not shown) to provide additional securing force; deformable material (not shown) within the cavity formed by opposing layers of the sleeve that deforms when inflated (or partially inflated) and which forms a rigid structure when the sleeve is decompressed and/or vacuumized, thereby rigidly and securely associating the biocompatible polymer (and comminuted decellularized fish skin particles and/or one or more therapeutics) with the wound; a plurality of base materials 1102 (and comminuted decellularized fish skin particles and/or one or more therapeutics); and/or a liner (not shown) of base material (and comminuted decellularized fish skin particles and/or one or more therapeutics).

In one or more embodiments, the outer cover 1108 is made of and/or includes flexible material (e.g., thermoplastic elastomer, elastomer, spandex, lycra, polymer aerogels, and the like); adjustable straps (not shown); and/or heat reflective material configured to retain and/or reflect body heat back to the associated wound and/or nearby tissue/body (e.g., polymer aerogels; radiant barrier fabrics; aluminum foil-fabric laminates; metallized thin film-fabric laminates; direct-metallized fabrics; insulated fabrics such as, for example, quilted or baffled fabrics; and/or other heat reflective and/or insulated materials known in the art).

In one or more embodiments, the comminuted decellularized fish skin particles 1004, 1104 of the disclosed temporary bandages 1000, 1100 provides one or more advantageous and/or salubrious properties. For example, the comminuted decellularized fish skin particles may act as a hemostatic agent to reduce and/or stop the loss of blood from a wound.

Hemostatic Applications

The decellularized fish skin has significantly better hemostatic properties compared to the coagulation agent and other biologic products tested (Table 1). The hemostatic effects of the decellularized fish skin may not be emitted directly through thrombin activation but may be due to the collagen content of the decellularized fish skin. Platelets in the blood may bind directly to collagen with collagen-specific glycoprotein surface receptors. Collagen binding to glycoprotein may start a signaling cascade that activates, among other things, platelet integrins that mediate tight binding of platelets to the decellularized fish skin. This process results in an adhesion of platelets to the site of injury. The hemostatic properties of the decellularized fish skin are promising as a novel hemostatic agent with the hemostatic effects derived from other means or in addition to thrombin activation. With a long shelf life and light packaging the product could be an essential addition to the first aid hemostatic combat kit, independent of the Acute Care Cover for the Severely Injured Limb (ACCSIL) device.

To establish blood coagulation in the combat theatre a variety of substances increasing blood coagulation have been added to different vehicles in the purpose of applying these devices to a heavily bleeding area. Intuitively it seems a good idea to coagulate blood in this way. The substances used include thrombin, which is a blood platelet activator and chitosan which binds the calcium ion (Ca²⁺). Ca²⁺ is an important factor in clot formation. The hemostatic characteristics of a device designed for covering large or small tissue defects or tissue destruction sustained by high-velocity ammunition, shrapnel, or the heat off an improvised explosive device (IED) should be present but not at the price of possible further harm to the injury site or hindrance of future operations on the wounded area.

In one embodiment, the Lee White blood clotting test was conducted in triplicate and hemostatic effects of wound treatment products such as Oasis® (Smith & Nephew Inc.) and Matristem® (Acell Inc.) compared to that of decellularized fish skin (Kerecis). This was done at a fixed concentration of 3.5 mg/mL blood. Total volume was 1 mL of whole blood. 1 NIH unit/mL of thrombin was used as positive control. Blood without any additives was used as a negative control (NEG). Blood coagulation time was recorded when the blood was completely clotted. Statistical analysis was performed with the Wilcoxon rank sum test, a.k.a. the Mann Whitney U test. The results are displayed in Table 1 below.

In another embodiment, the Lee White blood clotting test was performed for six healthy individuals in the age range of 23-45 years. The hemostatic effects of the following agents in 1 cm²/1 mL blood were tested: Kerecis™ Omega3, Oasis®, Matristem®, and Thrombin (1 NIH unit/mL) for positive control. Blood without any additives was used as a negative control (NEG). Blood was drawn from participants in an empty vacutainer and test material added. The glass tubes were tilted slowly to about 45° every 10 seconds, and when no blood was seen leaking to the side, the time was recorded. The results are illustrated in Table 2 below.

Decellularized fish skin induced significantly faster coagulation than mammalian derived products (p≤0.0001). Also, the decellularized fish skin supersedes coagulation by thrombin, which is used in the human natural hemostatic pathway. The decellularized fish skin show an average of about 2 minutes faster blood coagulation than thrombin and roughly 3 minutes faster coagulation than mammalian-derived membrane products.

In some embodiments, decellularized fish skin may coagulate blood more than 2 minutes faster than thrombin, more than 1 minute and 45 seconds faster than thrombin, more than 1 minute and 30 seconds faster than thrombin, more than 1 minute and 15 seconds faster than thrombin, more than one minute faster than thrombin, more than 45 seconds faster than thrombin, more than 30 seconds faster than thrombin, more than 15 seconds faster than thrombin, or at any rate faster than thrombin. In one embodiment, decellularized fish skin may coagulate blood at the same rate as thrombin.

In some embodiments, faster coagulation may not be due to thrombin activation alone, but may relate to platelet binding directly to glycoprotein surface receptors in the decellularized fish skin. In another embodiment, faster coagulation may be due to a combination of thrombin activation and platelet binding directly to glycoprotein surface receptors in the decellularized fish skin.

Tables 1 and 2 below include results of one-tailed t tests (Wilcoxon rank sum test) performed between different groups of hemostatic agents shown in FIGS. 1 and 2, respectively.

TABLE 1

| Comparison | p value | Significance |
|---|---|---|
| Kerecis - NEG | 0.009 | <0.01 |
| Kerecis - Oasis ® | 0.00005 | <0.0001 |
| Kerecis - Matristem ® | 0.00009 | <0.0001 |
| Kerecis - Thrombin | 0.004 | <0.01 |
| Oasis ® - NEG | 0.3 | — |
| Oasis ® - Thrombin | 0.06 | — |
| Oasis ® - Matristem ® | 0.4 | — |
| Matristem ® - NEG | 0.3 | — |
| Matristem ® - Thrombin | 0.092 | — |

TABLE 2

| Comparison | p value | Significance |
|---|---|---|
| Kerecis - NEG | 0.018 | <0.05 |
| Kerecis - Oasis ® | 0.00002 | <0.0001 |
| Kerecis - Matristem ® | 0.00017 | <0.001 |
| Kerecis - Thrombin | 0.009 | <0.01 |
| Oasis ® - NEG | 0.7 | — |
| Oasis ® - Thrombin | 0.11 | — |
| Oasis ® - Matristem ® | 0.3 | — |
| Matristem ® - NEG | 0.7 | — |
| Matristem ® - Thrombin | 0.092 | — |

In addition to blood loss, blast injury often results in complicated soft-tissue losses requiring a targeted approach for optimal outcomes. A key element includes early debridement and application of a cover to accelerate healing and prevent bacterial contamination and infection. Up to one quarter of wounds endured in combat become infected and require higher-Echelon care; furthermore, infection following blast-related injuries remains a major cause of morbidity and mortality in injured servicemen. Consequently, there is a need to improve the care closer to the point of injury to positively affect the definitive reconstructive options available after retrieval.

In one or more embodiments, the anti-viral and anti-bacterial properties of comminuted decellularized fish skin particles act to prevent bacterial and/or viral infection at the wound site, thereby decreasing potential complications (e.g., infection of the wound) and/or increasing the diversity of applications and or circumstances where temporary bandages may be used.

Additionally, the anti-inflammatory (or inflammatory regulating) properties of Omega3 PUFAs within comminuted decellularized fish skin help regulate inflammation at the wound site, which in some embodiments helps to stabilize and/or protect the tissue.

As a non-limiting example, comminuted decellularized fish skin particles may be applied directly to a dirty (e.g., not pre-cleaned or debrided) wound in the field (e.g., Echelon I treatment). The comminuted decellularized fish skin particles can be used as a standalone treatment or as part of a temporary bandage. One or more salubrious properties of the comminuted decellularized fish skin described above allow, in some embodiments, for the stabilization and/or protection of the underlying wound. It should be appreciated that once applied to the wound site, the fish skin particles can be later removed via debridement of associated tissue or by irrigation of the wound site. In the event that any particles remain at the wound site, the decellularized fish skin particles can be absorbed safely by the body without an inflammatory response or need for surgical removal thereafter.

As provided above, one beneficial aspect of the comminuted decellularized fish skin particles is their ability to stabilize and/or preserve a wound. Once applied to the wound, such as the dirty, non-debrided wound in the field (e.g., Echelon I treatment), the treatment allows the injured individual to be transported longer distances or for greater periods of time with a preserved wound site. In a similar fashion, the comminuted decellularized fish skin particles can be applied to a clean and/or debrided wound prior to application of a fresh temporary bandage at, for example, Echelon II treatment, so the wound can be stabilized and/or preserved while the wounded individual is transferred elsewhere for additional treatment (e.g., to Echelon III, IV, or V treatment, as appropriate). The salubrious properties of the comminuted decellularized fish skin may also allow, in some embodiments, for increased autograft-take by preparing the wound better than cadaver skin or other known materials (at, for example, Echelon IV or V treatment).

The comminuted decellularized fish skin particles disclosed above (or temporary bandages associated therewith) may be used to stabilize and/or protect traumatic wounds (e.g., gunshot wounds, stabbing wounds, wounds received from explosive blasts and/or shrapnel, crushed or severed limbs/appendages, and the like), burns, and/or amputations (e.g., voluntary, emergency, or resulting from a traumatic injury). The comminuted decellularized fish skin particles can impart numerous salubrious effects (e.g., hemostatic effects, anti-viral effects, anti-bacterial effects, inflammatory response regulatory effects, etc.), and when used in a temporary bandage, the base material can act to moderate the moisture content/environment of the wound to prevent drying out and/or deterioration of tissue while the outer cover provides physical support and in some embodiments pressure and/or structure to protect the wound during transit.

Kits for Stabilizing and/or Protecting a Wound

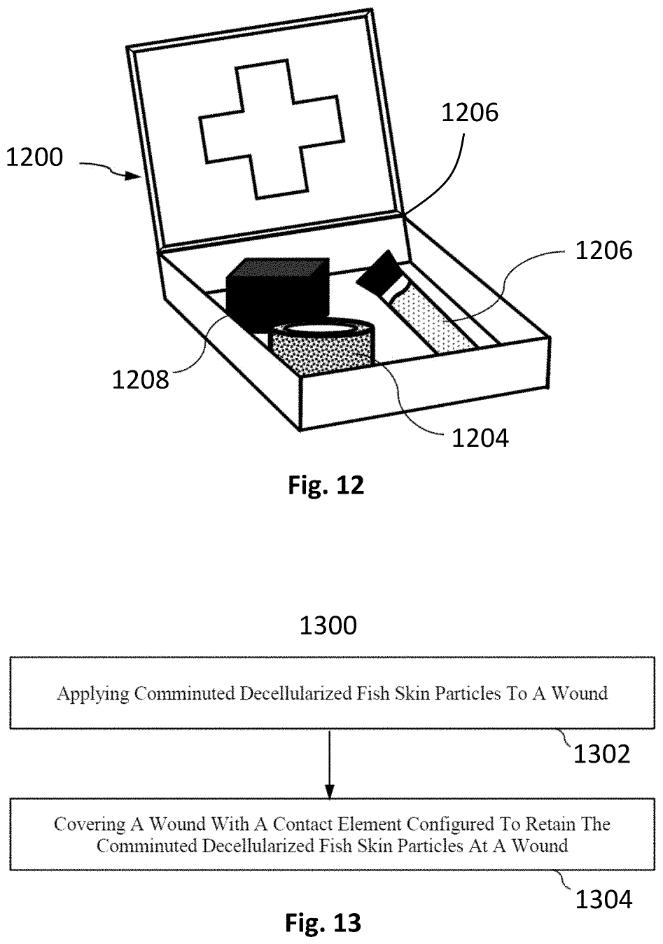

As should be appreciated, any of the foregoing or other temporary bandages (or components thereof) can be included in a kit. For example, as shown in FIG. 12, a kit 1200 for stabilizing and/or protecting a wound can include an outer container 1202 that houses, includes, or contains (i) a contact element 1204 configured to interface with a wound, (ii) comminuted decellularized fish skin particles 1206 for placement on a wound and to be retained on a wound by the contact element 1204, and (iii) an outer cover 1208 configured to retain the contact element 1204 and the comminuted decellularized fish skin particles 1206 at a wound.

With reference to the contact element 1204, the contact element 1204 can be or include a contact layer 1204 and can include any material or property discussed above with respect to the contact layer 1204. Alternatively, the contact element 1204 can be a material that acts to retain the comminuted decellularized fish skin particles 1206 at the wound. This can include, for example, gauze, a compressive sleeve, an adhesive bandage, padding, adherent wrap, or other wound dressing known in the art.

Any kit or temporary bandage disclosed herein can be used in any number of methods for stabilizing and/or protecting a wound. FIG. 13 illustrates an exemplary method 1300 for stabilizing and/or protecting a wound. The method 1300 includes applying comminuted decellularized fish skin particles to a wound (act 1302) and covering the wound with a contact element configured to retain the comminuted decellularized fish skin particles at the wound (act 1304). Variations of the method 1300 can additionally include an act of applying an outer cover to the contact element to secure the contact element to a partial or whole limb comprising the wound. Additionally, or alternatively, variations of method 1300 of applying the comminuted decellularized fish skin to the wound includes preserving tissue conditions at or near the wound.

In embodiments, applying the comminuted decellularized fish skin particles at the wound (act 1302) includes compacting or packing the comminuted decellularized fish skin particles into a ball or other mass/shape and inserting the ball or mass/shape into the wound. A clinician may form the comminuted decellularized fish skin particles into the ball based on a geometry of a wound, such as a tunneling/undermined wound.

In some embodiments, the kits (or components thereof) can be used for stabilizing, covering, and/or initializing the wound healing process in tunneled/undermined wounds or other traumatic wounds, including, for example, for the local management of bleeding wounds (e.g., cuts, lacerations, and abrasions) and/or the temporary management of severely bleeding or hemorrhaging wounds.

In some embodiments, additional advantages can be realized through the utilization of systems, kits, and/or methods that incorporate the two types of decellularized fish skin products (e.g., sheet-based and comminuted decellularized fish skin) together for complicated soft tissue wounds. These two types of fish skin can be used in combination with each, serving a different application purpose. For example, deep, asymmetric, and undermined wounds can be filled with comminuted decellularized fish skin before being secured with sheets for optimal wound healing, bleeding control, and protection against infection during transit to a higher Echelon facility. The secondary cover with sheet-based decellularized fish skin sheets protects the comminuted product during dressing changes and adds a bacterial and (additional) hemostatic barrier during transit. Consequently, injured individuals can begin healing while they await extraction to a healthcare facility, resulting in better quality wound beds for subsequent grafting.

Additionally, kits having comminuted decellularized fish skin can provide one or more surgical benefits, including, for example: providing wounds an initial treatment approach that will control bleeding, stabilize the wound bed, begin the skin regeneration process, and provide microbial control; simplify the treatment options for tunneling and undermining wounds that traditional materials are not physically optimized to address; fill deep sacral and pressure wounds, allowing smaller flaps to be applied and improving the likelihood of flap success; and temporize wounds in preparation for autografting and/or skin flap creation.

### EXAMPLES

The following examples as set forth herein are intended for illustrative purposes only and are not intended to limit the scope of the disclosure in any way, as many variations thereof are possible without departing from the spirit and scope of the disclosure.

### Example 1

Wound healing properties of particularized and intact fish skin sheets in a splinted excisional mouse model to investigate the capacity of particularized fish skin to induce granulation and facilitate healing of deep subcutaneous splinted excision wounds in mice.

Wound healing in a mouse is fundamentally different to that of humans as it primarily occurs via contraction. By splinting the wound, the repair process is then dependent on epithelialization, cellular proliferation and angiogenesis, which closely mirror the biological processes of human wound healing.

Male C57BL/6 are used. Each mouse will receive two wounds, enabling the application of both the decellularized fish skin and standard of care treatment (positive control) on the same animal, so each animal is its own control. After carefully shaving and depilating the mouse's back, a sterile 4 mm biopsy is used to punch the outline of two circular patterns for the wound on either side of the mouse's midline at the level of the shoulders. Serrated forceps are used to lift the skin in the middle of the outline and iris scissors to create a full thickness wound that extends through the subcutaneous tissue, including the panniculus carnosus and excise the circular piece of tissue.

The process is repeated for the wound on the other side of the midline.

The silicon “donut”-like splint is 10 mm in diameter, 0.5 mm thick with 5 mm hole in the middle. Plastic protective coating is removed from each side of the silicone splint. Cyanoacrylate adhesive is applied to one side of a silicone splint. The splint is centered over the wound and anchored with interrupted 6-0 nylon sutures to ensure proper positioning. A ruler is placed below the splints and a photo is taken using a macro lens.

Mice are divided into two groups with three time points; D7, D14 and D21:

Group A: only the treatment wound is filled with comminuted decellularized fish skin; the control wound will not be filled. Afterward, both wounds are covered with a transparent, occlusive dressing (such as OpSite).

Group B: only the treatment wound is filled with small pieces of intact decellularized fish skin; the control wound will not be filled. Afterward, both wounds are covered with a transparent, occlusive dressing (such as OpSite).

Anesthesia and analgesia is provided according to animal facility recommendation, with the exception that NSAIDs will not be used as they can inhibit inflammation and therefore influence the healing process.

The wounds are visually inspected, photographed, and their size measured at every dressing change, 1-2 times a week. If fish skin is incorporated into the wound bed within 1 week and there is still available space, additional fish skin is inserted to mimic clinical practice.

Mice are followed for up to 21 days with groups sacrificed at days 3, 7, 14, and 21. After euthanasia by cervical dislocation, the splint is removed and wide, full excision around and under the wound area is created. The tissue is incubated for further diagnosis by histology to examine inflammation, granulation, and quality of healing.

qPCR is performed to quantify expression of relevant wound healing-related genes (e.g., VEGF, IL-1b, eNOS, iNOS). Size of wounds is determined by using ImageJ or other comparable software.

Treatment and control wounds are randomized to right or left side of the mouse.

Parametric and non-parametric statistical analysis are performed as appropriate. Statistical analysis will also be performed on LDI data and other bioassay results. Paired Student's t-test and ANOVA F-test are conducted to assess the significance of the difference in histology among the treatment groups. Post-hoc analysis is adjusted for multiple testing. P values <0.05 are considered significant.

Results include wounds treated with comminuted decellularized fish skin being stabilized and showing initialization of wound healing at the wound site earlier and/or more robustly than the control, decreased inflammation at the wound site, and an increased abundance, concentration, and/or half-life of healing-related transcripts.

Results also include the wounds treated with sheet-based decellularized fish skin as having less incidence of infection and a greater amount of cellular ingrowth (granulation), decreased inflammation, and increased quality of healing compared to controls.

### Example 2

Hemostatic properties of fish skin in a porcine femoral artery hemorrhage model to investigate the hemostatic properties of decellularized fish skin. The aim of this study is to demonstrate that particularized fish skin is more effective in controlling hemorrhage than the control (standard of care) product without any apparent side effect. A 50% reduction in post treatment blood loss is considered to be clinically significant.

For all surgical procedures, supportive measures to include anesthesia, analgesia, fluid maintenance, warming etc. are taken to maintain physiologic homeostasis (unless contraindicated in the experimental protocol), and minimize any pain or distress in the animals. Following anesthesia, Yorkshire cross-bred male pigs weighing 34 kg to 44 kg are intubated and a catheter placed in the ear vein to administer maintenance fluid.

TABLE 3
Inclusion/exclusion criteria for the porcine hemorrhage model.

| Inclusion Criteria | Exclusion Criteria |
|---|---|
| Hematocrit: 27%-40% | Unexpected death due to anesthesia or technical |
| | error. |
| Platelet: ≥2 K/mm³ | Persistent low MAP (<55 mg Hg) at the baseline. |
| PT: ≤14 s | Significant blood loss (>300 mL) because of |
| | surgical complication or error before femoral |
| | injury. |
| PTT: ≤25 s | Pretreatment blood loss (during 45 s of free |
| | bleeding) of <10 mL/kg or >25 mL/kg. |
| Fibrinogen: ≥100 | Persistent hypotension and unresponsive to |
| mg/dL | fluid resuscitation despite no bleeding. |
| Body weight: 33-44 kg | |
| Gender: male | |

An arterial hemorrhage model is undertaken.

Right carotid artery is cannulated and connected to a pressure transducer for recording of blood pressure. Right jugular vein is catheterized for administering resuscitation fluid during hemorrhage and wound treatment.

Midline laparotomy and cystostomy is performed, and abdomen is closed by suturing and stapling the skin. An incision of 10 cm is made in the groin area close to the femoral artery, and 5 cm of the artery are dissected free from surrounding tissue with cauterization and ligation of small arterial branches.

Artery is bathed with 2% lidocaine to dilute it to its normal diameter. Fluid maintenance is then discontinued. After a 5-10-minute stabilization period the artery proximally and distally is clamped and a 6 mm diameter arteriotomy on the anterior surface of the vessel is made 2-3 cm from the bottom of the groin. Clamps are released and free bleeding is allowed for 45 seconds. Blood is collected by suction.

The pigs are separated into four groups: (a) comminuted decellularized fish skin, (b) sheet-based decellularized fish skin, (c) standard of care (i.e., Combat gauze, positive control), and (d) regular gauze (negative control).

Immediately after the free bleeding starts, the respective products are opened and packed into the wound and covered with a laparotomy sponge or gauze: (a) comminuted decellularized fish skin compacted to form an adherent ball; a sponge is used to stabilize the product in place and press it against the wound; (b) 3×7 cm sheet of decellularized fish skin is folded in half and pressed up against the wound by a sponge; (c) a sponge is used to press combat gauze against the wound; (d) a sponge is used to press standard gauze against the wound.

Manual pressure is kept for 3 min to stop the bleeding. Skin flaps are pulled over the sponge without clamping or creating additional pressure on the test materials. Fluid resuscitation is then started, with infusion of 500 mL of Hextend (6% HES in balanced electrolyte solution+glucose) via the jugular vein catheter to raise and maintain the MAP between 60 mm Hg and 65 mm Hg. Afterwards, fluid resuscitation is continued with max 10 L LR solution. After compression, pressure is slowly released, and hemostasis observed for 3 minutes. Initial hemostasis is considered to be achieved if no bleeding is apparent during this period. All shed blood is collected continuously and time to hemostasis recorded. The volume of blood lost is calculated and reported as post-treatment blood loss. Pigs are monitored up to 2.5 h or until death. Survival time is recorded, and final blood samples collected. Surviving pigs may be scanned with CT. Legs of surviving pigs are flexed to test stability of hemostasis. Product is removed to examine status of clots and patency of vessel. Animals are euthanized with intravenous injection as per institutional standards and tissue samples collected for histology. Gross necropsy is performed on vital organs. Histologic slides are prepared for H&E staining.

The primary end points measured are post treatment blood loss, bleeding/hemostasis time (time period necessary for bleeding to stop), MAP, survival time, and percentage survival. The secondary end points include hemoglobin, hematocrit, platelet counts, pH, lactate, base deficit, and coagulation values (e.g., PT, aPTT, fibrinogen, and TEG parameters).

The statistical analysis of LDI data and other bioassay results are performed as needed. ANOVA F-test is conducted to assess the significance of the difference among the treatment groups in % re-epithelialization and molecular changes over the time course. Post-hoc analysis are conducted using Tukey's honestly significant difference (HSD) to adjust for multiple testing. Non-parametric tests are incorporated as appropriate for corresponding data. P values <0.05 are considered significant.

Results include at least a 50% reduction in post-treatment blood loss in groups (a) and (b) with group (a) having the largest reduction in post-treatment blood loss and highest survival rate.

### Example 3

Deep soft tissue wound repair properties of fish skin in swine to investigate the capacity of comminuted decellularized fish skin to induce granulation tissue formation in deep, undermined, subcutaneous excision wounds in pigs and/or to shorten the time and provide a more favorable wound bed for Split Thickness Skin Graft.

Following debridement, one approach is negative pressure wound therapy (NPWT) to promote blood flow to the wound, control edema, and reduce the presence of proteases, thus leading to increased granulation and revascularization of the wound bed. While NPWT is a traditional therapy for complex wound healing, it is not always practical for use during prolonged field care and rapid evacuation to higher Echelons of care. Noted disadvantages of NPWT include, for example: inability to accurately control applied pressure in geometrically challenging wounds or wounds near or at anatomically sensitive areas where adhesive seals are hard to obtain, bleeding (which may be difficult to assess due to obstruction from the dressing), skin irritation, infection, pain or discomfort, ingrowth of granulation tissue into dressing materials, as well as machine- or device-related technical problems. Some NPWT machines, for example, are not mobile, rely on electricity, require continuous proximity to the patient to be effective, and add to the complexity of patient transfers during retrieval.

Juvenile castrated male Duroc swine are used to minimize potential interference from the estrogen cycle and to reduce animal aggressiveness. For all surgical procedures, supportive measures to include anesthesia, analgesia, fluid maintenance, warming etc. are taken to maintain physiologic homeostasis (unless contraindicated in the experimental protocol), and minimize any pain or distress in the animals.

Each animal will receive four wounds, two on each side of the flank. The wounds are created using a combination of sharp and blunt excision within the perimeter of approximately 3″×3″. The depth is to subcutaneous tissue, fascia and muscle, with some undermining. Wounds are treated within 1 hour after wound creation with: (a) SOC Dressing (e.g., normal saline, sulfamylon, or similar [wet to dry]); (b) comminuted decellularized fish skin with a sheet of decellularized fish skin as a cover affixed with sutures or staples; (c) NPWT with VAC device (KCI); (d) comminuted decellularized fish skin with a sheet of decellularized fish skin as a cover intact fish skin cover and also NPWT.

Wounds will then be covered with non-adherent dressings and secured with neoprene garments. Animals are recovered and monitored.

Garments and dressings are taken down with wounds examined and evaluated for readiness for grafting at post-injury days 2/3, 4/5, 6/7, or every 2-3 days until wound is deemed ready for grafting by experienced clinician. When prepared, wounds are autografted with a 3:1 or 4:1 widely meshed autograft and dressed.

After grafting, wounds are examined 1-2 times per week and monitored for graft take and healing through at least Day 28 or until re-epithelialization, if sooner. At the end of the study, the animals are euthanized according to the facility's standard and recommended procedure.

At all of the above time points for the experiment, wounds are photographed, imaged using Laser Doppler Imaging (LDI) to assess perfusion, swabbed for subsequent cultures/microbiome analyses, and 2 punch biopsies are taken (each 2 mm)—one preserved in formalin and one in AllProtect Reagent (or flash frozen).

The endpoints include: (a) day post-injury when wound bed is ready for grafting; (b) graft take at Day 5-7 after grafting; (c) time until full epithelialization; (d) scarring/aesthetic appearance of healed wound at 4 weeks post graft; (e) histologic analyses to examine inflammation, re-epithelialization, and ECM (H&E and Masson's Trichrome); (f) expression of selected wound healing related genes, quantified by mRNA analysis.

Pigs are followed for 6-8 weeks post grafting, so total study time period is 9-12 weeks.

Clinical assessors are blinded.

ANOVA F-test are conducted to assess the significance of the difference among the treatment groups in % re-epithelialization and molecular changes over time course. Post-hoc analysis are conducted using Tukey's honestly significant difference (HSD) to adjust for multiple testing. Non-parametric tests are incorporated as appropriate for corresponding data. P values <0.05 are considered significant.

Results include groups (b) and (d) outperforming the positive control (group (a)) and doing at least as well as, or in some cases significantly better than, group (c).

### Abbreviated List of Defined Terms

To assist in understanding the scope and content of the foregoing written description and appended claims, a select few terms are defined directly below.

As used herein, the term “base material” may include any material known in the art that may act as a vehicle for therapeutics and which may additionally, or alternatively, enable and/or passively regulate moisture at and/or surrounding a wound.

The term “biocompatible polymer” refers to a polymer material which is not harmful to a human body. A biocompatible polymer includes any synthetic or natural polymer material which does not release substances harmful to a human body and which does not cause side effects such as skin stimulation—even when coming in direct contact with and a wound site—or any other negative influence on the human body.

The degrees of “Echelon,” as used herein, refer to locations and/or types of medical attention provided to military personnel. Echelon I refers to self-aid and buddy-aid treatments as well as combat medic treatments administered in the battlefield or at locations remote from Echelon II personnel/facilities. Echelon II refers to advanced trauma care by physicians, physician's assistants, or other qualified medical personnel, and Echelon II care is often administered at a field hospital. Echelon III refers to care provided at the corps level and typically includes reconstructive and definitive surgery to save life, limb, and eyesight; this care may be provided at a field hospital with the necessary equipment. Echelon IV refers to complex surgery and prolonged convalescence (e.g., greater than two weeks) and is generally provided at regional, permanent hospitals. Echelon V refers to injuries and/or procedures that require extensive rehabilitation and convalescent care; Echelon V treatments are administered at continental US permanent hospitals. Although the foregoing Echelon system is particularly relevant to military personnel and treatment scenarios, the Echelon system may also be analogized, as appropriate, to treatment locations and/or types of treatment scenarios in a civilian and/or local law enforcement scenario.

The term “wound” as used herein is intended to encompass tissue injuries generally. Thus, the term “wound” includes those injuries that cause, for example, cutting, tearing, and/or breaking of the skin such as lacerations, abrasions, incisions, punctures, avulsions, or other such injuries. Wounds may be described by any of the size, shape, or magnitude of the wound. For example, a paper cut is exemplary of a small, straight incision of relatively little magnitude, whereas a concussive blast resulting in a major laceration covering one or multiple body parts is exemplary of a relatively larger wound of greater magnitude. Each of the foregoing examples, however, fall within the scope of the term “wound,” as used herein.

The term “wound” additionally includes damage to underlying tissue, such as that caused by traumatic injury. As such, the term “wound” is intended to include a combination of multiple different wounds. For example, a traumatic amputation caused by an explosive blast may generally be referred to as a wound even though it is a compilation of a host of different lacerations, abrasions, avulsions, lesions, and punctures. Additionally, any underlying tissue damage resulting from the aforementioned explosive blast may further be encompassed within the understanding of this reference to a wound. The term “wound” is also intended to encompass tissue injuries caused by burns (e.g., thermal and/or chemical burns). Further, the term “wound” is also intended to encompass injuries resulting from, for example, diabetic foot ulcers, venous leg ulcers, surgical operations, pressure ulcers, and other causes.

Further, the wounds amenable to treatment by the wound treatment and methods disclosed herein include injuries that can be located in any site, including internal, interfacial, external, interstitial, extracorporeal, and/or intracorporeal. Examples of wounds suitable for coverage with the scaffold material include cuts, gashes, open wounds, tissue rupture, Decubitus, Dermatitis, lesions, chronic wounds, battlefield wounds, necrotic wounds, acute, chronic, traumatic, lacerations, abrasions, contusions, necrotizing fascitis, toxic epidermal necrolysis, pressure wounds, venous insufficiency ulcers, arterial ulcers, diabetic or neuropathic ulcers, pressure ulcers, mixed ulcers, burn wounds, Mucormycosis, Vasculitic wounds, Pyoderma, gangrenosum, and equivalents, and/or combinations thereof, known by persons skilled in the art. Treatment of wounds in human and animal subjects are contemplated.

In certain embodiments, the wound treatment and methods disclosed herein is used for abdominal wall reconstruction, for example to repair hernias. For example, in repairing a hernia, a surgeon will make an incision near the location of the hernia. For an inguinal hernia, the incision is made just above the crease where the abdomen meets the thigh. To repair an umbilical hernia, it is made close to the navel. If the hernia has occurred at the site of a previous operation, the incision from that surgery is reopened. Surgery proceeds in much the same way, regardless of where the incision is made. The hernia sac is carefully opened and the intestine or other tissue is placed back inside the abdomen. The weakened area is repaired and reinforced with a synthetic mesh or a suture that pulls the abdominal muscle tissue back together.

A “traumatic wound,” as used herein refers to any wound resulting from physical injury that damages both the skin and underlying tissue. A gunshot wound is one non-limiting example of a traumatic wound, as it causes a puncture (i.e., a break) in the skin and ruptures or otherwise damages underlying tissue. As another non-limiting example, a concussive or explosive blast generally results in traumatic wound(s). Many, but not all, of the wounds received during wartime may be described as traumatic wounds due to the nature of war and war-related injuries. A “traumatic wound” can include hemorrhaging wounds, wounds with exposed bone and/or tendons, severe burns, deep tissue wounds (e.g., asymmetrical deep-tissue wounds), and/or large surface area wounds.

By providing wound treatment according to the disclosed embodiments, the problems of existing wound treatments being subject to shear forces and difficult to apply to complex wound geometries are advantageously addressed. The wound treatment and method embodiments of the present disclosure allow a clinician to provide a scaffold material to facilitate cellular ingrowth and neovascularization while better conforming to a wound bed and, in embodiments, cooperating with a sheet of scaffold material to provide a substrate for wound healing.

This disclosure provides various examples, embodiments, and features which, unless expressly stated or which would be mutually exclusive, should be understood to be combinable with other examples, embodiments, or features described herein.

In addition to the above, further embodiments and examples include the following:

1. A wound treatment comprising: particles of decellularized fish skin, wherein a predetermined percentage of at least a first portion of the particles of decellularized fish skin have a greatest dimension within a predetermined size threshold maximum and a minimum size threshold that is effective to preserve a matrix structure of the decellularized fish skin and to promote cellular regenerative ingrowth into a wound.

2. The wound treatment of 1, wherein the predetermined size threshold is greater than 1 mm.

3. The wound treatment of 1, wherein the predetermined size threshold is between 1 mm and 1.39 mm, or wherein the predetermined size threshold is between 1.4 and 1.99 mm.

4. The wound treatment of 1, wherein the predetermined size threshold is between 2 mm and 2.8 mm.

5. The wound treatment of 1, wherein the predetermined size threshold is less than 1 mm.

6. The wound treatment of 1, wherein the predetermined size threshold is between 1 mm and 2 mm.

7. The wound treatment of 1, wherein the predetermined size threshold is over 2 mm.

8. The wound treatment according to any or a combination of 1-7 above or 9-10 below, wherein the predetermined size threshold pertains to a length and/or width of the particles of the decellularized fish skin, or wherein the predetermined size threshold does not pertain to a thickness of the particles of the decellularized fish skin.

9. The wound treatment according to any or a combination of 1-8 above or 10 below, wherein the predetermined percentage of the particles of decellularized fish skin having the greatest dimension within the predetermined size threshold and the minimum size threshold is 75% or more of the particles.

10. The wound treatment according to any or a combination of 1-9 above, wherein a second predetermined percentage of at least a second portion of the particles of decellularized fish skin have a greatest dimension within a second predetermined size threshold maximum and/or a second minimum size threshold, and wherein the second predetermined size threshold maximum is different than the predetermined size threshold maximum of the first portion of the particles and/or the second minimum size threshold is different than the minimum size threshold of the first portion of the particles.

11. A wound treatment method, the method comprising: providing particles of decellularized fish skin, a predetermined percentage of at least a first portion of the particles of decellularized fish skin have a greatest dimension within a predetermined size threshold maximum and a minimum size threshold that is effective to preserve a matrix structure of the decellularized fish skin and to promote cellular regenerative ingrowth into a wound; applying the particles of decellularized fish skin to a wound bed; and covering the wound bed with a dressing.

12. The wound treatment method according to any or a combination of 11 above or 13-22 below, further comprising the steps: preparing the wound bed for treatment before the applying of the particles of the decellularized fish skin to the wound bed; securing the dressing; and checking the wound bed for integration of the shredded, decellularized fish skin particles.

13. The wound treatment method according to any or a combination of 11-12 above or 14-22 below, wherein the dressing is a non-adherent dressing comprising a synthetic non-woven or cotton woven dressing.

14. The wound treatment method according to any or a combination of 11-13 above or 15-22 below, wherein applying the particles of decellularized fish skin to the wound bed includes conforming the particles of decellularized fish skin to a shape of the wound bed.

15. The wound treatment method according to any or a combination of 11-14 above or 15-22 below, further comprising: moistening the particles of decellularized fish skin with a liquid before application to obtain moistened particles of decellularized fish skin.

16. The wound treatment method according to any or a combination of 12, wherein the checking of the wound bed for integration takes place up to two weeks after application of the particles of decellularized fish skin to the wound bed.

17. The wound treatment method according to any or a combination of 11-16 above or 18-22 below, wherein the particles of decellularized fish skin define a substantially rectangular configuration.

18. The wound treatment method according to any or a combination of 11-17 above or 19-22 below, wherein the particles of decellularized fish skin are provided in a package, the package being configured to receive a liquid.

19. The wound treatment method according to any or a combination of 15, wherein the moistened particles of decellularized fish skin are formed into a paste prior to application to the wound bed.

20. The wound treatment method according to any or a combination of 11-19 above or 21-22 below, wherein the particles of decellularized fish skin are used in combination with a sheet-based decellularized fish skin scaffold.

21. The wound treatment method according to any or a combination of 11-20 above or 22 below, wherein the sheet-based decellularized fish skin scaffold is applied over the wound bed after application of the particles of decellularized fish skin.

22. The wound treatment method according to any or a combination of 11-21, wherein the providing of the particles of decellularized fish skin includes providing a second predetermined percentage of at least a second portion of the particles of decellularized fish skin having a greatest dimension within a second predetermined size threshold maximum and/or a second minimum size threshold, and wherein the second predetermined size threshold maximum is different than the predetermined size threshold maximum of the first portion of the particles and/or the second minimum size threshold is different than the minimum size threshold of the first portion of the particles

23. A method of providing a wound treatment, comprising the steps of: providing one or more sheets of decellularized fish skin; and shredding or grinding the one or more sheets of decellularized fish skin into particles of decellularized fish skin such that a predetermined percentage of at least a first portion of the particles of decellularized fish skin have a greatest dimension within a predetermined size threshold maximum and a minimum size threshold that is effective to preserve a matrix structure of the decellularized fish skin and to promote cellular regenerative ingrowth into a wound.

In addition to the above, further embodiments and examples include the following:

1. A temporary wound treatment comprising comminuted decellularized fish skin in particle form.

2. The temporary wound treatment according to any or a combination of 1 above or 2-12 below, wherein the temporary wound treatment comprises a temporary bandage configured and arranged to deliver the comminuted decellularized fish skin in particle form to a wound, the temporary bandage comprising: a contact layer configured to interface with a wound and to retain the comminuted decellularized fish skin at a wound; and an outer cover associated with the contact layer, the outer cover configured to retain the contact layer at a wound.

3. The temporary wound treatment according to 2 above, further comprising a base material associated with the contact layer and carrying the comminuted decellularized fish skin particles.

4. The temporary wound treatment according to 3 above, wherein the comminuted decellularized fish skin particles are releasable from the base material in a time-dependent manner.

5. The temporary wound treatment according to any or a combination of 1-4 above or 6-12 below, wherein the comminuted decellularized fish skin particles are smaller than about 1 cm in diameter, smaller than about 0.1 cm in diameter, smaller than about 10 mm in diameter, smaller than about 1 mm in diameter, smaller than about 0.1 mm in diameter, smaller than about 10 μm in diameter, smaller than about 1 μm in diameter, or combinations thereof.

6. The temporary wound treatment according to any or a combination of 1-5 above or 7-12 below, further comprising a compression element associated with the outer cover, the compression element configured to conform the outer cover to a shape of a wound.

7. The temporary wound treatment according to any or a combination of 1-5 above or 8-12 below, further comprising a compression element associated with the outer cover, the compression element configured to conform the outer cover to a shape of a partial or whole limb comprising a wound.

8. The temporary wound treatment according to any or a combination of 6-7 above, wherein the compression element comprises a sleeve having an inflatable bladder.

9. The temporary wound treatment according to 8, further comprising a base material associated with the contact layer and carrying the comminuted decellularized fish skin particles, wherein the base material is disposed at a bottom or a peripheral wall of the sleeve.

10. The temporary wound treatment according to any or a combination of 1-9 above or 11-12 below, wherein the comminuted decellularized fish skin particles comprise partially processed, comminuted decellularized fish skin particles.

11. The temporary wound treatment according to 10 above, wherein the partially processed, comminuted decellularized fish skin particles have been treated with one or more enzymes to reduce a rigidity of the comminuted decellularized fish skin particles.

12. The temporary wound treatment according to any or a combination of 11, wherein at least a portion of extracellular matrix material within the partially processed, comminuted decellularized fish skin particles have been cleaved by the one or more enzymes, increasing one or more of a ductility or an elasticity of the partially processed, comminuted decellularized fish skin particles.

13. A kit for stabilizing and/or protecting a wound, comprising: a container including comminuted decellularized fish skin in particle form for placement on or in a wound and to be retained at a wound site by a contact element.

14. The kit according to any or a combination of 13 above or 15-18 below, wherein the container further comprises: a contact element configured to interface with a wound and to retain the comminuted decellularized fish skin in particle form at a wound; and an outer cover configured to retain the contact element and the comminuted decellularized fish skin in particle form at a wound.

15. The kit according to any or a combination of 13-14 above or 16-18 below, wherein the container further comprises a base material for carrying the comminuted decellularized fish skin in particle form and for associating with the contact element.

16. The kit according to any or a combination of 13-15 above or 17-18 below, wherein the container further comprises one or more therapeutics comprising analgesics, anesthetics, cytokines, growth factors, hemostatic agents, antibiotics, antifungals, hydrating compounds, or combinations thereof.

17. The kit according to any or a combination of 13-16 above or 18 below, wherein the comminuted decellularized fish skin particles are smaller than about 1 cm in diameter, smaller than about 0.1 cm in diameter, smaller than about 10 mm in diameter, smaller than about 1 mm in diameter, smaller than about 0.1 mm in diameter, smaller than about 10 μm in diameter, smaller than about 1 μm in diameter, or combinations thereof.

18. The kit according to any or a combination of 14-17 above, wherein the container further comprises a compression element configured to associate with the outer cover, the compression element conforming the outer cover to a shape of a wound and/or a partial or whole limb comprising a wound when the compression element is associated with the outer cover.

19. A method for stabilizing and/or protecting a wound, comprising applying comminuted decellularized fish skin in particle form to a wound.

20. The method according to any or a combination of 19 above or 21-28 below, further comprising: covering a wound with a contact element configured to retain the comminuted decellularized fish skin particles at a wound; and applying an outer cover to the contact element to secure the contact element to a partial or whole limb comprising a wound.

21. The method according to any or a combination of 19-20 above or 22-28 below, wherein applying the comminuted decellularized fish skin to a wound includes preserving tissue conditions at or near a wound.

22. The method according to 21 above, wherein preserving tissue conditions at or near a wound includes one or more of decreasing a loss of damaged tissue or increasing a likelihood that an affected area comprising a wound can be rehabilitated.

23. The method according to 21 or 22 above, wherein preserving tissue conditions at or near a wound includes moderating a moisture content of a wound to prevent a wound from drying out or from causing deterioration of tissue at or near a wound.

24. The method according to any or a combination of 19-23 above or 25-28 below, wherein applying the decellularized fish skin to a wound includes increasing hemostasis at a wound.

25. The method according to any or a combination of 19-24 above or 26-28 below, wherein applying the decellularized fish skin to a wound includes decreasing pain associated with a wound.

26. The method according to any or a combination of 19-25 above or 27-28 below, wherein applying comminuted decellularized fish skin particles to a wound comprises compacting the comminuted decellularized fish skin particles into a ball and inserting the ball into a wound.

27. The method according to any or a combination of 19-26 above or 28 below, wherein a wound is a deep tissue wound, a hemorrhaging wound, or a wound having exposed bone and/or tendon.

28. The method according to any or a combination of 19-27 above, wherein applying comminuted decellularized fish skin in particle form to a wound does not promote ingrowth of cellular tissue within the decellularized fish skin in particle form.

29. A bandage for treatment of an unclean and/or non-debrided wound site, the bandage comprising comminuted decellularized fish skin in particle form

30. The bandage according to any or a combination of 29 above or 31-33 below, wherein the comminuted decellularized fish skin in particle form is rehydrated prior to application to a wound site.

31. The bandage to any or a combination of 29-30 above or 32-33 below, further comprising a covering to secure the comminuted decellularized fish skin in particle form at a wound site.

32. The bandage to any or a combination of 29-31 above or 33 below, wherein a wound site comprises a deep wound and the comminuted fish skin in particle form is compacted into a deep wound.

33. The temporary wound treatment to any or a combination of 1-12 above, wherein the particle form is configured to minimize cellular scaffolding at a wound site during temporary wound treatment.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

It is to be understood that not necessarily all objects or advantages may be achieved under an embodiment of the disclosure. Those skilled in the art will recognize that the wound treatment and methods for making the same may be embodied or carried out in a manner that achieves or optimizes one advantage or group of advantages as taught herein without achieving other objects or advantages as taught or suggested herein.

The skilled artisan will recognize the interchangeability of various disclosed features. Besides the variations described herein, other known equivalents for each feature can be mixed and matched by one of ordinary skill in this art to prepare a wound treatment and utilize a method for making the same under principles of the present disclosure. The skilled artisan will understand that the features described herein may be adapted to other types of wound treatments and healthcare applications generally.

Although this disclosure describes certain exemplary embodiments and examples of a wound treatment, it will be understood by those skilled in the art that the present disclosure extends beyond the specifically disclosed wound treatment embodiments to other alternative embodiments and/or uses of the disclosure and obvious modifications and equivalents thereof. It is intended that the present disclosure should not be limited by the disclosed embodiments described above and may be extended to other applications that may employ the features described herein.

1. A wound treatment comprising:
particles of decellularized fish skin,
wherein a predetermined percentage of at least a first portion of the particles of decellularized fish skin have a greatest dimension within
a predetermined size threshold maximum and
a minimum size threshold that is effective to preserve a matrix structure of the decellularized fish skin and to promote cellular regenerative ingrowth into a wound.

2. The wound treatment of claim 1, wherein the predetermined size threshold is greater than 1 mm.

3. The wound treatment of claim 1, wherein the predetermined size threshold is between 1 mm and 1.39 mm, or
wherein the predetermined size threshold is between 1.4 and 1.99 mm.

4. The wound treatment of claim 1, wherein the predetermined size threshold is between 2 mm and 2.8 mm.

5. The wound treatment of claim 1, wherein the predetermined size threshold is less than 1 mm.

6. The wound treatment of claim 1, wherein the predetermined size threshold is between 1 mm and 2 mm.

7. The wound treatment of claim 1, wherein the predetermined size threshold is over 2 mm.

8. The wound treatment of claim 1, wherein the predetermined size threshold pertains to a length and/or width of the particles of the decellularized fish skin, or wherein the predetermined size threshold does not pertain to a thickness of the particles of the decellularized fish skin.

9. The wound treatment of claim 1, wherein the predetermined percentage of the particles of decellularized fish skin having the greatest dimension within the predetermined size threshold and the minimum size threshold is 75% or more of the particles.

10. The wound treatment of claim 1, wherein a second predetermined percentage of at least a second portion of the particles of decellularized fish skin have a greatest dimension within
a second predetermined size threshold maximum and/or a second minimum size threshold, and
wherein the second predetermined size threshold maximum is different than the predetermined size threshold maximum of the first portion of the particles and/or the second minimum size threshold is different than the minimum size threshold of the first portion of the particles.

11. A wound treatment method, the method comprising:
providing particles of decellularized fish skin, a predetermined percentage of at least a first portion of the particles of decellularized fish skin have a greatest dimension within a predetermined size threshold maximum and a minimum size threshold that is effective to preserve a matrix structure of the decellularized fish skin and to promote cellular regenerative ingrowth into a wound;
applying the particles of decellularized fish skin to a wound bed; and
covering the wound bed with a dressing.

12. The wound treatment method of claim 11, further comprising the steps:
preparing the wound bed for treatment before the applying of the particles of the decellularized fish skin to the wound bed;
securing the dressing; and
checking the wound bed for integration of the shredded, decellularized fish skin particles.

13. The wound treatment method of claim 11, wherein the dressing is a non-adherent dressing comprising a synthetic non-woven or cotton woven dressing.

14. The wound treatment method of claim 11, wherein applying the particles of decellularized fish skin to the wound bed includes conforming the particles of decellularized fish skin to a shape of the wound bed.

15. The wound treatment method of claim 11, further comprising:
moistening the particles of decellularized fish skin with a liquid before application to obtain moistened particles of decellularized fish skin.

16. The wound treatment method of claim 12, wherein the checking of the wound bed for integration takes place up to two weeks after application of the particles of decellularized fish skin to the wound bed.

17. The wound treatment method of claim 11, wherein the particles of decellularized fish skin define a substantially rectangular configuration.

18. The wound treatment method of claim 11, wherein the particles of decellularized fish skin are provided in a package, the package being configured to receive a liquid.

19. The wound treatment method of claim 15, wherein the moistened particles of decellularized fish skin are formed into a paste prior to application to the wound bed.

20. The wound treatment method of claim 11, wherein the particles of decellularized fish skin are used in combination with a sheet-based decellularized fish skin scaffold.

21. The wound treatment method of claim 20, wherein the sheet-based decellularized fish skin scaffold is applied over the wound bed after application of the particles of decellularized fish skin.

22. The wound treatment method of claim 11, wherein the providing of the particles of decellularized fish skin includes providing a second predetermined percentage of at least a second portion of the particles of decellularized fish skin having a greatest dimension within a second predetermined size threshold maximum and/or a second minimum size threshold, and
wherein the second predetermined size threshold maximum is different than the predetermined size threshold maximum of the first portion of the particles and/or the second minimum size threshold is different than the minimum size threshold of the first portion of the particles

23. A method of providing a wound treatment, comprising the steps of:
providing one or more sheets of decellularized fish skin; and
shredding or grinding the one or more sheets of decellularized fish skin into particles of decellularized fish skin such that a predetermined percentage of at least a first portion of the particles of decellularized fish skin have a greatest dimension within
a predetermined size threshold maximum and
a minimum size threshold that is effective to preserve a matrix structure of the decellularized fish skin and to promote cellular regenerative ingrowth into a wound.
